(19) United States
(12) Patent Application Publication
(10) Pub. No.: US 2025/0114642 A1
(43) Pub. Date: Apr. 10, 2025

(54) GANTRY CONFIGURED FOR TRANSLATIONAL MOVEMENT

(71) Applicant: Mevion Medical Systems, Inc., Littleton, MA (US)

(72) Inventors: Robert Silva, Nashua, NH (US); Gerrit Townsend Zwart, Durham, NH (US); James Cooley, Boxborough, MA (US)

(21) Appl. No.: 18/726,204

(22) PCT Filed: Dec. 28, 2022
(86) PCT No.: PCT/US2022/054153

§ 371 Date: Jul. 2, 2024

Related U.S. Application Data
(60) Provisional application No. 63/296,610, filed on Jan. 5, 2022.

(51) Int. Cl. A61N 5/10

(52) U.S. Cl. CPC A61N 5/1081; A61N 5/1049; A61N 2005/1061; A61N 2005/1087

(57) ABSTRACT

An example system includes a gantry including a beamline structure configured to direct a particle beam from an output of a particle accelerator toward an irradiation target at a treatment position. The beamline structure includes magnetic bending elements to bend the particle beam along at least part of a length of the beamline structure. A mount, on which at least part of the beamline structure is held, is configured to enable translational movement of at least part of the beamline structure relative to the irradiation target.

### CROSS-REFERENCE TO RELATED APPLICATION

This application claims priority to U.S. Provisional Application No. 63/296,610, which was filed on Jan. 5, 2022. U.S. Provisional Application No. 63/296,610 is hereby incorporated into this application by reference.

### TECHNICAL FIELD

This specification describes examples of particle therapy systems and gantries for use therewith.

### BACKGROUND

Particle therapy systems use a particle accelerator to generate a particle beam for treating afflictions, such as tumors. Particle therapy systems may use a gantry to direct the particle beam toward a patient from multiple angles. In some examples, a gantry includes a device that supports a radiation delivery apparatus during treatment.

### SUMMARY

An example system includes a gantry including a beamline structure configured to direct a particle beam from an output of a particle accelerator toward an irradiation target at a treatment position. The beamline structure includes magnetic bending elements to bend the particle beam along at least part of a length of the beamline structure. A mount, on which at least part of the beamline structure is held, is configured to enable translational movement of at least part of the beamline structure relative to the irradiation target. The system may include one or more of the following features, either alone or in combination.

The translational movement may include movement along a longitudinal dimension of the gantry. The translational movement may include movement toward or away from the particle accelerator. The system may include the particle accelerator and the mount may be configured to enable movement of the particle accelerator along with movement of the at least part of the beamline structure. The mount may be configured to enable movement of an entirety of the beamline structure relative to the irradiation target. The mount may be configured to enable movement of the entirety of the beamline structure along a longitudinal dimension of the gantry. The mount may be configured to enable movement of the entirety the beamline structure toward or away from the particle accelerator along at least part of a beamline of the particle beam.

The translational movement may cause the at least part of the beamline structure to move away from the particle accelerator and to produce an air gap between the at least part of the beamline structure and the particle accelerator. The particle beam may traverse the air gap from the particle accelerator to the at least part of the beamline structure. The at least part of the beamline structure that is subject to translational movement may include a first part of the beamline structure. The beamline structure may include the first part and a second part. The translational movement may cause the first part to move away from the second part and to produce an air gap between the first part and the second part. The particle beam may traverse the air gap. The second part, which is not subject to translational movement, may be attached to the particle accelerator and need not be movable relative to the particle accelerator.

The at least part of the beamline structure that is subject to the translational movement may include an output channel. The output channel may include magnetic dipoles arranged in series to bend the particle beam by at least 90°. The gantry may include a ring structure on which the output channel is mounted for rotation around the irradiation target. The translational movement of the at least part of the beamline structure may be parallel to an axis of rotation about which the output channel rotates on the ring structure. The translational movement may be for at least 30 centimeters. The translational movement may be between 30 centimeters and 1 meter of movement. The translational movement may exceed 1 meter of movement.

The system may include an imaging system that is movable relative to the irradiation target and a control system to control the mount or the at least part of the gantry to move the at least part of the beamline structure away from a location proximate to the irradiation target, and to control movement of the imaging system toward that location. A couch or seat for holding the irradiation target may be configured to remain stationary during movement of the imaging system and during movement of the mount or the at least part of the beamline structure.

The mount may be a first mount and the system may include a second mount configured to enable rotational movement of the imaging system relative to the irradiation target. The control system may be configured to control movement of the imaging system by controlling translational movement of the second mount. The imaging system may be rotatable around an axis of rotation defined, for example, by the second mount. The translational movement of the second mount may be parallel to this axis of rotation. The control system may be configured to control movement of the imaging system away from the location proximate to the irradiation target and to control the first mount or the at least part of the beamline structure to move the at least part of the beamline structure toward that location. The couch for holding the irradiation target may be configured to remain stationary during movement of the imaging system and during movement of the first mount or the at least part of the beamline structure. The control system may be configured to control movement of the imaging system by controlling translational movement of the second mount. The imaging system may be rotatable around the axis of rotation defined by the second mount and the translational movement of the second mount may be parallel to this axis of rotation.

The first mount on which at least part of the beamline structure is held may include one or more rails. The one or more rails may be moveable or the at least part of the beamline structure may be movable along the one or more rails. The first mount on which at least part of the beamline structure is held may include one or more rollers or wheels connected to the at least part of the beamline structure.

The at least part of the beamline structure that is subject to translational movement may include a nozzle. The nozzle may be for holding at least one of an energy degrader or a collimator. The system may include an imaging system that is movable relative to the irradiation target and a control system to control a mount holding the nozzle or the nozzle to move the nozzle away from a location proximate to the irradiation target, and to control movement of the imaging system toward that location. A couch or seat for holding the irradiation target may be configured to remain stationary during movement of the imaging system and during movement of the mount or the nozzle. The mount holding the nozzle may include a rail-mounted drawer. The mount holding the nozzle may be configured to move the nozzle telescopically.

An example method may be implemented on a particle therapy system. The method may be implemented using one or more processing devices. Operations in the method may include receiving data representing a size of a target beam field and controlling translational movement of at least part of a beamline structure of a gantry in the particle therapy system relative to an irradiation target based on the data. The beamline structure may be configured to direct a particle beam from an output of a particle accelerator toward the irradiation target. The beamline structure may include magnetic bending elements to bend the particle beam along at least part of a length of the beamline structure. The operations may include controlling the particle accelerator to apply particle beam to the irradiation target at different translational positions of the at least part of the beamline structure based on the data. A couch holding the irradiation target may remain stationary during the translational movement of the at least part of the beamline structure and application of the particle beam. The method may include one or more of the following features, either alone or in combination.

The method may include controlling rotational movement of at least part of the beamline structure relative to the irradiation target. The couch may be configured to remain stationary during the rotational movement of the at least part of the beamline structure. The translational movement in the method may include movement of the at least part of a beamline structure along a longitudinal dimension of the gantry to discrete positions along the irradiation target. The translational movement in the method may include movement of the at least part of a beamline structure toward or away from the particle accelerator along at least part of a beamline of the particle beam.

The beamline structure may include an output channel. The output channel may include magnetic dipoles arranged in series to bend the particle beam by at least 90°. The gantry may include a ring structure on which the output channel is mounted for rotation around the irradiation target. The translational movement of the at least part of a beamline structure may be parallel to an axis of rotation about which the output channel rotates on the ring structure.

The method may include controlling movement of an imaging system based on the translational movement of the at least part of the beamline structure while the irradiation target is controlled or configured to remain stationary. The at least part of the beamline structure may be controlled to move out of a predefined position and the imaging system may be controlled to move to the predefined position following movement of the at least part of gantry. The imaging system may be controlled to move out of the predefined position and the beamline structure may be controlled to move back to the predefined position following movement of the imaging system out of the predefined position. The size of a target beam field may be greater than a size of a predefined beam field defined, at least in part, by the gantry absent the translational movement of the at least part of gantry. The size of the target beam field may be at least 1.5 times the size of the predefined beam field. The size of the target beam field may be at least twice the size of the predefined beam field. The size of the target beam field is at least five times the size of the predefined beam field.

Any two or more of the features described in this specification, including in this summary section, may be combined to form implementations not specifically described in this specification.

Control of the various systems described herein, or portions thereof, may be implemented via a computer program product that includes instructions that are stored on one or more non-transitory machine-readable storage media and that are executable on one or more processing devices (e.g., microprocessor(s), application-specified integrated circuit(s), programmed logic such as field programmable gate array(s), or the like). The systems described herein, or portions thereof, may be implemented as an apparatus, method, or a medical system that may include one or more processing devices and computer memory to store executable instructions to implement control of the stated functions. The devices, systems, and/or components described herein may be configured, for example, through design, construction, composition, arrangement, placement, programming, operation, activation, deactivation, and/or control.

The details of one or more implementations are set forth in the accompanying drawings and the following description. Other features and advantages will be apparent from the description and drawings, and from the claims.

### DESCRIPTION OF THE DRAWINGS

FIG. 1 is a diagram showing a partially transparent perspective view of an example particle therapy system having an example gantry of the type described herein.

FIG. 2 is a cut-away, side view of components of the particle therapy system shown in FIG. 1, including the example gantry.

FIG. 3 is a cut-away, close-up, side view of components included in in a nozzle and an example beamline structure that may be part of the gantry shown in FIG. 1.

FIG. 4 is a cut-away, close-up, side view of components included in an example nozzle and beamline structure that may be part of the gantry shown in FIG. 1.

FIG. 5 is an illustration of a front view of an example scanning magnet configured to scan a particle beam in two orthogonal dimensions.

FIG. 6 is a cut-away, close-up, side view of components included in an example nozzle and beamline structure that may be part of the gantry shown in FIG. 1.

FIG. 7 is an illustration of a front view of an example scanning magnet configured to scan a particle beam in a single dimension.

FIG. 8 is an illustration of a front view of an example scanning magnet configured to scan a particle beam in a single dimension.

FIG. 9 is a cut-away, close-up, side view of components included in an example nozzle and beamline structure that may be part of the gantry shown in FIG. 1.

FIG. 10 is a cut-away, close-up, side view of components included in an example nozzle and beamline structure that may be part of the gantry shown in FIG. 1.

FIG. 11 is an illustration of a front view of an example superconducting scanning magnet configured to scan a particle beam in two orthogonal dimensions.

FIG. 12a is an illustration of a front view of an example superconducting scanning magnet configured to scan a particle beam in a single dimension; and FIG. 12b is an illustration of a front view of an example superconducting scanning magnet configured to scan a particle beam in a single dimension orthogonal to the dimension of FIG. 12a.

FIG. 13 is a drawing showing a perspective view of an example configurable collimator that may be part of the particle therapy system of FIG. 1.

FIG. 14 is a drawing showing a front, partially-transparent view of the configurable collimator of FIG. 13.

FIG. 15 is a drawing showing a perspective, partially-transparent view of the configurable collimator of FIGS. 13 and 14.

FIG. 16 is a block diagram of an example treatment space that is configured to house all or part of the particle therapy system of FIG. 1.

FIG. 17 is a graph showing example horizontal (x) and vertical (y) particle beam envelopes produced using the example gantry described herein.

FIG. 18 is a graph showing an example achromatic lattice design for the beamline of the example gantry described herein.

FIG. 19 is a graph showing results produced by scanning the particle beam in the horizontal (x) and vertical (y) planes using the example gantry described herein.

FIG. 20 is a cut-away, side view of components in an example particle accelerator that may be used with the particle therapy system described herein.

FIG. 21 is a perspective view of an example energy degrader.

FIG. 22 is a front, cut-away view of an example superconducting magnet that may be used as a scanning magnet in the particle therapy system of FIG. 1.

FIG. 23 is a cut-away view of part of an example superconducting tape that may be would into a coil and used as a scanning magnet in the particle therapy system of FIG. 1,

FIG. 24 is a block diagram side view of an example particle therapy system in which at least part of a gantry is configured for translational movement.

FIG. 25 is a block diagram side view an example particle therapy system in which a gantry beamline structure is configured for translational movement.

FIG. 26 is a block diagram side view an example particle therapy system in which part of the gantry beamline structure is configured for translational movement.

FIG. 27 is a block diagram side view an example particle therapy system in which the gantry beamline structure and particle accelerator are configured for tandem translational movement.

FIG. 28 is a block diagram side view of an example particle therapy system in which at least part of the gantry is configured for translational movement using a mount that includes rollers or wheels.

FIGS. 29 and 30 are block diagram side views of an example particle therapy system in which a gantry nozzle is configured for translational movement.

FIGS. 31 and 32 are block diagram side views of an example particle therapy system in which a gantry nozzle is configured for telescopic translational movement.

FIGS. 33 and 34 are block diagram side views of an example particle therapy system in which gantry translational movement in the longitudinal dimension is used to effectively extend the beam field achievable by the particle therapy system.

FIG. 35 is a flowchart showing an example process for controlling gantry translational movement in the longitudinal dimension to effectively extend the beam field achievable by the particle therapy system.

Like reference numerals in different figures indicate like elements.

### DETAILED DESCRIPTION

Described herein are example particle therapy systems that may house the patient and the accelerator in the same space. An example system includes a particle accelerator that may be, but is not limited to, a synchrocyclotron that has low radiation leakage and that is small enough to fit within a standard linear accelerator (LINAC) vault. The system also includes a medical gantry configured to deliver a charged particle beam, such as protons or ions, output from the accelerator to treat tumors or other conditions in a patient. The gantry includes a beamline structure to direct the particle beam from the accelerator to a treatment position and to deliver the particle beam to the treatment position. The beamline structure includes magnetics, such as one or more magnetic dipoles and one or more magnetic quadrupoles, to direct the particle beam toward the treatment position. To enable delivery of the particle beam in the same space that is used for treatment, particularly in relatively small spaces such as a standard LINAC vault, at least some of the magnetics in the beamline structure are configured to bend the particle beam at right angles or at obtuse angles. In an example, the magnetics are configured and arranged to bend the particle beam by 90° or greater.

Implementations of the particle therapy system described herein also include a mount on which at least part of the gantry is held. The mount is configured to enable automated and motorized translational movement of at least part of the gantry relative to a predefined reference, such as the irradiation target, the treatment position, or the particle accelerator. For example, the mount may include rollers or one or more rails on which all or part of the beamline structure is mounted. The accelerator may also be mounted on, or connected to, the roller(s) or rail(s) to enable tandem movement with the beamline structure. The translational movement may enable at least part of the gantry, with or without the accelerator, to move in a longitudinal dimension—for example, parallel to its rotational axis. For example, the beamline structure can be moved out of the treatment position and one or more imaging systems moved into its place.

An imaging system may capture an image of a target, such as a tumor in a patient, at the treatment position. Following image capture, the imaging system may be moved back to its original position, which is out of the way of the gantry and the treatment path. At least part of the gantry (e.g., at least part of the beamline structure—which may be or include a nozzle) may then be moved back into the treatment position. At that position, the gantry may be used to treat the target at the treatment position. During these movements of the gantry and the imaging system, the patient at the treatment position may remain stationary. For example, the patient may be positioned on a couch, which does not move during movement of the gantry and the imaging system. For example, the patient himself may not move on the couch. Reducing opportunities for patients to move on the couch reduces the chances that a treatment will be delivered incorrectly, or that the couch may need to be repositioned to compensate for movements.

Furthermore, the translational movement of at least part of the gantry can extend the beam field of the system. In an example, the beam field includes the maximum extent that a particle beam can be moved across a plane over or parallel to a treatment position for a given position of the gantry without moving the patient. By moving the gantry as described herein, the size of the beam field can be increased, thereby supporting treatments such as craniospinal irradiations, in which a patient's entire brain and spinal column are treated, without moving the patient or by moving the patient less than would be required using gantries not capable of translational movement.

Implementations of the particle therapy system described herein also combine the functionality of large-aperture superconducting magnets with the use of upstream scanning magnets to make the particle therapy system relatively compact. Although compact in construction, the example particle therapy system is configured to enable beam focusing, beam scanning, beam bending, and beam rotation as described below.

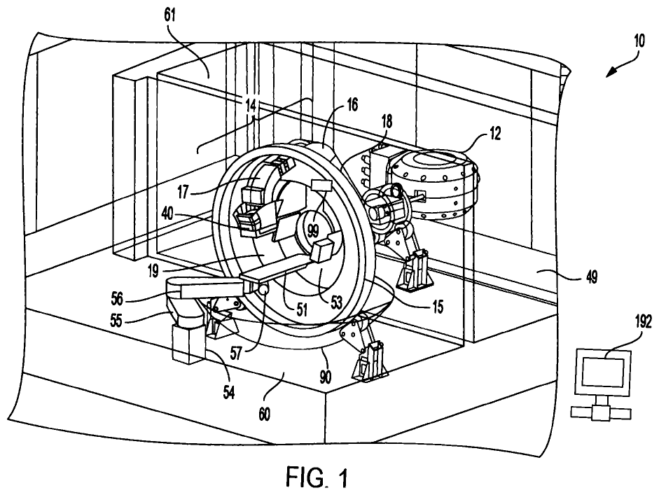

FIG. 1 shows components of an example of a particle therapy system 10 of the type described in the preceding paragraphs. As shown in FIG. 1, particle therapy system 10 includes a particle accelerator 12, examples of which are described herein. In this example, particle accelerator 12 is a synchrocyclotron having a superconducting electromagnetic structure that generates a maximum magnet field strength of 2.5 Tesla (T) or more or 3 T or more. In this regard, a superconductor is an element or metallic alloy such as niobium-tin (Nb₃Sn) which, when cooled below a threshold temperature, loses most, if not all, electrical resistance. As a result, current flows through the superconductor substantially unimpeded. Superconducting coils, therefore, are capable of conducting larger currents in their superconducting state than ordinary wires of the same size. Because of the high amounts of current that they are capable of conducting, superconducting coils are particularly useful in particle therapy applications.

An example synchrocyclotron is configured to output protons or ions as a monoenergetic particle beam having an energy level of 150 MegaElectronvolts (MeV) or more. The example synchrocyclotron has a volume of 4.5 cubic meters (m³) or less and a weight of 30 Tons (T) or less. Due to its size, this type of particle accelerator is referred to as “compact”. However, as described herein, synchrocyclotrons or other types of particle accelerators having weights, dimensions, magnetic fields, and/or energy levels other than these may be used in particle therapy system 10.

Particle therapy system 10 also includes gantry 14. Gantry 14 includes ring-shaped or circular support structure 15 and a beamline structure 16. The combination of support structure 15 and beamline structure 16 may be referred to as a “compact gantry” due to its relatively small size. Beamline structure 16 includes an output channel 17 that mounts to support structure 15 and a conduit 18 that directs the particle beam to the output channel. Gantry 14 also includes one or more motors (not shown) for moving output channel 17 around support structure 15 relative to a treatment position 19. The treatment position may include a system isocenter where a patient may be positioned on a patient couch for treatment. In an example, the motors may move output channel 17 along a track on structure 15 resulting in rotation of output channel 17 relative to treatment position 19. In an example, the support structure to which output channel 17 is attached may rotate relative to treatment position 19, resulting in rotation of output channel 17 relative to the treatment position. In some implementations, the rotation enabled by gantry 14 allows output channel 17 to be positioned at any angle relative to the treatment position. For example, output channel 17 may rotate through 360° and, as such, output channel 17 may be positioned at 0°, 90°, 270°, and back to 0°/360° or any angle among these rotational positions.

As noted previously, beamline structure 16 is configured to direct a particle beam from accelerator 12 to treatment position 19. To this end, output channel 17 includes magnetics to bend the particle beam toward the treatment position. In addition, beamline structure 16 includes conduit 18 containing magnetics along the beamline that direct the particle beam from particle accelerator 12 to output channel 17.

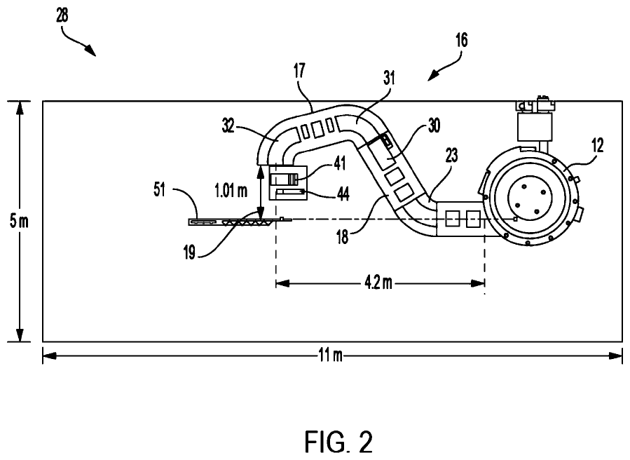

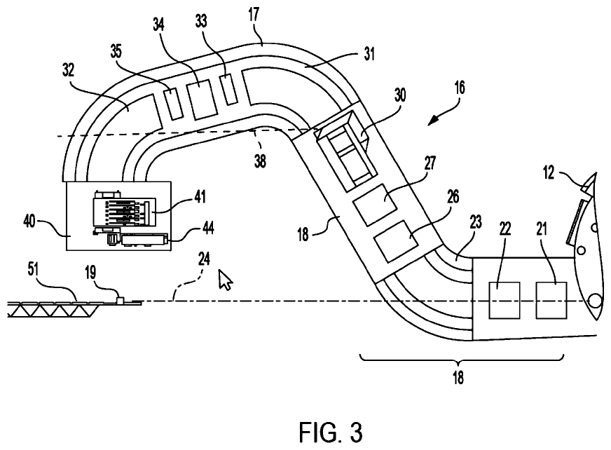

Referring to FIGS. 2 and 3, conduit 18 of example beamline structure 16 includes non-superconducting magnetic quadrupoles 21 and 22 and superconducting magnetic dipole 23. The beamline structure may also include an outer electromagnetically shielded shell. Referring to FIG. 3, magnetic quadrupoles 21 and 22 are configured to keep the particle beam focused and traveling straight or substantially straight—for example, a 5% or less deviation from straight—within beamline structure 16. Magnetic quadrupoles 21 and 22 are configured to focus the particle beam to maintain a substantially consistent cross-sectional area of the particle beam, for example, to within a tolerance of ±5%. Magnetic dipole 23 is configured to bend the particle beam toward output channel 17, as shown in the figures. In an example, magnetic dipole 23 may be configured to bend the particle beam anywhere in a range of 20° to 80° relative to horizontal 24. Generally, greater bend angles may reduce the distance between particle accelerator 12 and treatment position 19 or system isocenter, thereby reducing the space required to accommodate the gantry and, thus, the size of the particle therapy system. For example, replacing magnetic dipole 23 with one or more superconducting magnetic dipoles that bend the particle beam by more than 80°—for example, by 90° or more—may further reduce the distance from particle accelerator 12 to support structure 15 and, thus, to treatment position 19 and the isocenter.

In some implementations, higher-order magnetics may be used in place of, or in addition to, any magnetic quadrupoles described herein. For example, the beamline structure may include one or more magnetic sextupoles in place of, or in addition to, the magnetic quadrupoles. The magnetic sextupoles may be configured to keep the particle beam focused and traveling straight or substantially straight—for example, a 5% or less deviation from straight—within beamline structure 16. The magnetic sextupoles may also configured to maintain a consistent cross-sectional area of the particle beam, for example, to within a tolerance of ±5%. Also, sextupole magnets may correct for chromatic effect of a quadrupole magnet.

Referring to FIG. 3, in this example, conduit 18 of beamline structure 16 also includes two non-superconducting magnetic quadrupoles 26 and 27. Magnetic quadrupoles 26 and 27 are configured to keep the particle beam focused and traveling straight or substantially straight—for example, a 5% or less deviation from straight—within beamline structure 16. Magnetic quadrupoles 26 and 27 are configured to maintain a consistent cross-sectional area of the particle beam, for example, to within a tolerance of ±5%. As described previously, higher-order magnetics may be substituted for one or more of the magnetic quadrupoles to improve focusing.

Particle therapy system 10 also includes one or more scanning magnets 30 in the path of the particle beam and configured to move the particle beam across at least part of a beam field that covers all or part of (that is, at least part of) the irradiation target. Movement of the particle beam across the beam field results in movement across at least part of an irradiation target at a treatment position 19. The scanning magnets may be sized and configured to move the particle beam across a beam field having an area of 20 centimeters (cm) by 20 cm or greater, although system 10 is not limited to any particular beam field size or shape. For example, the scanning magnets may have an aperture of 20 cm by 20 cm or less or greater, although the scanning magnets are not limited to any particular aperture size. For example, the beam field may be rectangular, circular, square, or any shape supported by the scanning magnets.

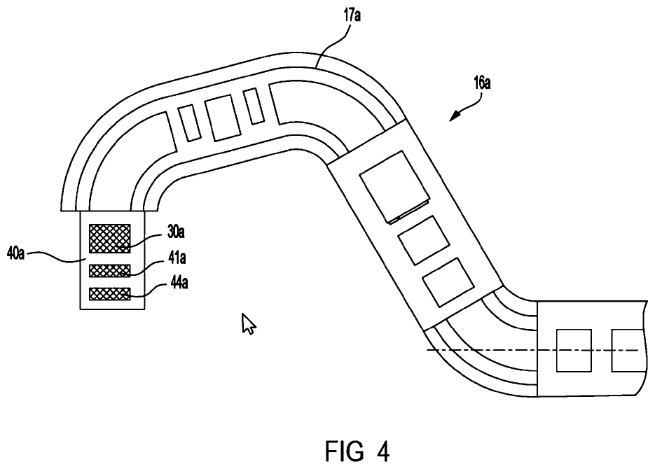

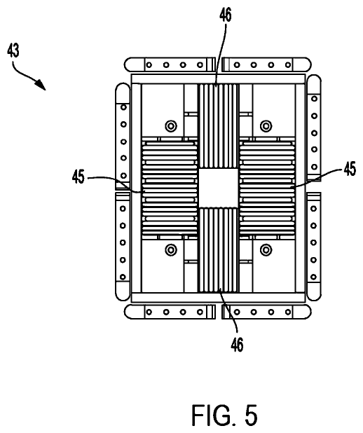

The scanning magnets may be located at different positions within the particle therapy system. For example, in beamline structure 16a shown in FIG. 4, which is a variant of beamline structure 16, all of the scanning magnets 30a may be located in nozzle 40a, along with energy degrader 41a and collimator 44a (both described below), on a path of the particle beam between output channel 17a and the treatment position. Referring to FIG. 5, an example scanning magnet 43 is controllable in two dimensions (e.g., Cartesian XY dimensions) to position the particle beam in those two dimensions and to move the particle beam across at least a part of an irradiation target. In this example, scanning magnet 43 includes a first set 45 of two coils, which control particle beam movement in the Cartesian X dimension of a defined coordinate system, and a second set 46 of two coils, which are orthogonal to the first set of two coils and which control particle beam movement in the Cartesian Y dimension. Control over movement of the particle beam may be achieved by varying current through one or both sets of coils to thereby vary the magnetic field(s) produced thereby. By varying the magnetic field(s) appropriately, the magnetic fields acts on the particle beam to move the particle beam in the X and/or Y dimension across a beam field and, thus, the irradiation target.

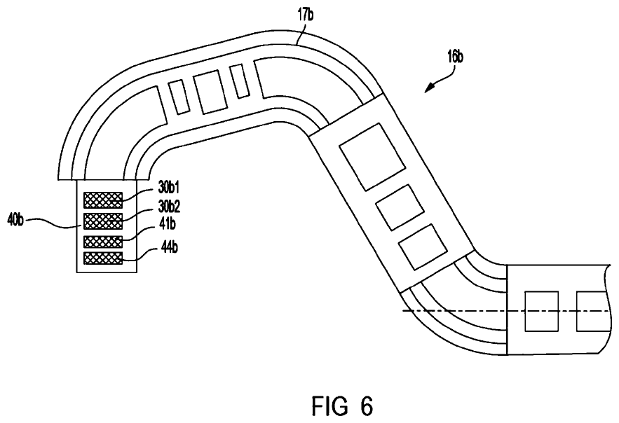

In some implementations there may be more than one scanning magnet. Implementations that include multiple scanning magnets that are at different points along the path of the particle beam and that are separated by air or structures such as magnets or beam-absorbing plates may be referred to as split scanning systems. For example, in beamline structure 16b shown in FIG. 6, which is a variant of beamline structure 16, there may be multiple—for example, two—scanning magnets 30b1 and 30b2 between the between output channel 17b and the treatment position. The scanning magnets may be located in nozzle 40b, along with energy degrader 41b and collimator 44b, on a path of the particle beam between output channel 17b and the treatment position. The scanning magnets may be at separate locations and separated by air or an energy-degrading structure. For example, in this implementation, a first scanning magnet 30b1 may move the particle beam in two dimensions (for example, Cartesian X and Y dimensions) and a second scanning magnet 30b2 may move the particle beam in two dimensions (for example, Cartesian X and Y dimensions). In this example, scanning magnets 30b1 and 30b2 may have the same construction and operation as the scanning magnet shown in FIG. 5. Each magnet 30b1 and 30b2 may move the beam partly, with the combined movements produced by the two magnets producing the desired movement specified in a treatment plan.

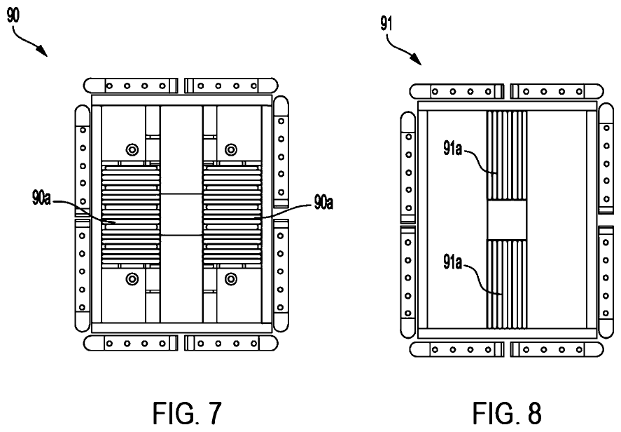

In a variant of the FIG. 6 implementation, scanning magnet 30b1 may move the particle beam in one dimension only (for example, the Cartesian X dimension) and scanning magnet 30b2 may move the particle beam in one dimension only (for example, the Cartesian Y dimension). One magnet 30b1 may be upstream of the other magnet 30b2 relative to the particle accelerator as shown in the figure. The two may be separated by air or an energy degrading structure as noted above. FIGS. 7 and 8 show example magnets 90 and 91, respectively, having orthogonal coils—coils 90a are orthogonal to coils 91a—to move the particle beam in different dimensions. In this example, scanning magnet 30b1 may be of the type shown in FIG. 7 and include a first set of coils 90a and scanning magnet 30b2 may be of the type shown in FIG. 8 and include a second set of coils 91a that are orthogonal to coils 90a. Each magnet 30b1, 30b2 may move the beam partly, with the combined movements produced by the two magnets producing the desired movement specified in a treatment plan.

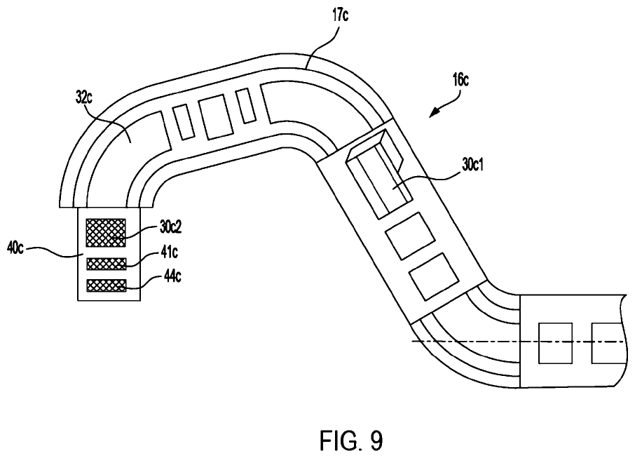

In some implementations, one or more—for example, all or fewer than all—of the scanning magnets may be located in the beamline structure. For example, in beamline structure 16c shown of FIG. 9, which is a variant of beamline structure 16, there may be multiple—for example, two—scanning magnets including a first scanning magnet 30c1 located within conduit 18 of beamline structure 16c and a second scanning magnet 30c2 located in nozzle 40c, along with energy degrader 41c and collimator 44c between output channel 17 and the treatment position. The first scanning magnet 30c1 may be located among the magnetics included in beamline structure 16c. For example, first scanning magnet 30c1 may be located within output channel 17c upstream of magnetic dipole 32c relative to the particle accelerator, or as shown in FIG. 9 first scanning magnet 30c1 may be located upstream of output channel 17c relative to the particle accelerator. In an example, first scanning magnet 30c1 may be configured to move the particle beam in two dimensions (for example, Cartesian X and Y dimensions) and second scanning magnet 30c2 may be configured to move the particle beam in two dimensions (for example, Cartesian X and Y dimensions). In this example, scanning magnets 30c1 and 30c2 may have the same construction and operation as the scanning magnet shown in FIG. 5. Each magnet 30c1 and 30c2 may move the beam partly, with the combined movements produced by the two magnets producing the desired movement specified in a treatment plan.

In a variant of the FIG. 9 implementation, first scanning magnet 30c1 may be configured to move the particle beam in one dimension only (for example, the Cartesian X dimension) and second scanning magnet 30c2 may be configured to move the particle beam in one dimension only (for example, the Cartesian Y dimension). In this example, scanning magnet 30c1 may include a first set of coils and scanning magnet 30c2 may include a second set of coils that are orthogonal to the first set of coils. Magnets 30c1 and 30c2 may have configurations like the magnets shown in FIGS. 7 and 8 in this example. Each magnet 30c1 and 30c2 may be configured to move the beam partly, with the combined movements produced by the two magnets producing the desired movement specified in a treatment plan.

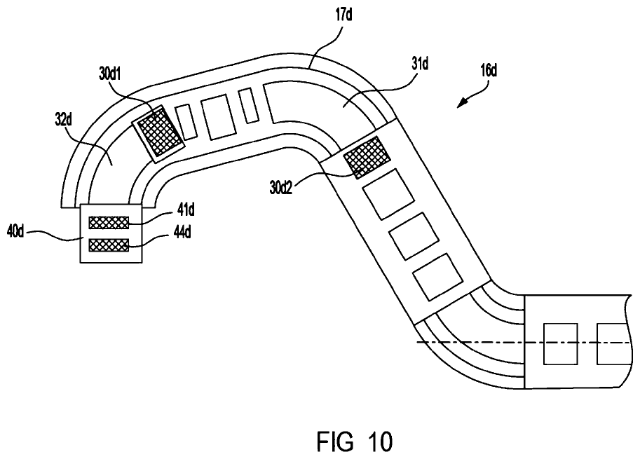

In some implementations, all of the scanning magnets may be located in the beamline structure upstream of the nozzle. As shown in the split scanning system of FIG. 10, both a first scanning magnet 30d1 and a second scanning magnet 30d2 may be located within beamline structure 16d upstream of the nozzle. No scanning magnets may be located in nozzle 40d, which includes energy degrader 41d and collimator 44d in this example. In other examples, there may be one or more scanning magnets also in the nozzle. First scanning magnet 30d1 and second scanning magnet 30d2 may be located among the magnetics included in beamline structure 16d upstream of the nozzle. For example, as shown in FIG. 10 first scanning magnet 30d1 may be located within output channel 17d upstream of magnetic dipole 32d relative to the particle accelerator, or the first scanning magnet may be located upstream of output channel 17d relative to the particle accelerator. Second scanning magnet 30d2 may be located upstream of first scanning magnet 30d1 relative to the particle accelerator. In the example shown in FIG. 10, second scanning magnet 30d2 precedes output channel 17d in the beamline. The scanning magnets may be at separate locations within the beamline structure and separated by magnetics, such as a dipole or quadrupole, and/or air within the beamline structure. The separate locations may include different points or locations in series along a path of the particle beam or length of the beamline structure. For example, as shown in FIG. 10, magnetic dipole 31d is between first scanning magnet 30d1 and second scanning magnet 30d1. In another example, scanning magnet 30d1 may be moved after magnetic dipole 32d such that both magnetic dipoles 31d and 32d are between scanning magnets 30d1 and 30d1. In another example, both scanning magnets 30d1 and 30d2 may be within output channel 17d and magnetic dipoles 31d and 32d may surround scanning magnets 30d1 and 30d2. In an example, first scanning magnet 30d1 may be configured to move the particle beam in two dimensions (for example, Cartesian X and Y dimensions) and second scanning magnet 30d2 may be configured to move the particle beam in two dimensions (for example, Cartesian X and Y dimensions), In this example, scanning magnets 30d1 and 30d2 may have the same construction and operation as the scanning magnet shown in FIG. 5. Each magnet 30d1 and 30d2 may move the particle beam partly, with the combined movements produced by the two scanning magnets producing the desired particle beam movement specified in a treatment plan.

In a variant of the FIG. 10 implementation, first scanning magnet 30d1 may be configured to move the particle beam in one dimension only (for example, the Cartesian X dimension) and second scanning magnet 30d2 may be configured to move the particle beam in one dimension only (for example, the Cartesian Y dimension). In this example, scanning magnet 30d1 may include a first set of coils and scanning magnet 30d2 may include a second set of coils that are orthogonal to the first set of coils. Magnets 30d1 and 30d2 may have configurations like the magnets shown in FIGS. 7 and 8 in this example. Each magnet 30d1 and 30d2 may be configured to move the beam partly, with the combined movements produced by the two magnets producing the desired movement specified in a treatment plan.

In some implementations, there may be more than two scanning magnets located within the beamline structure and/or located between the output of the output channel and the treatment position. For example, there may be three or more scanning magnets located at various separate locations within the beamline structure. For example, there may be three or more scanning magnets located at various separate locations between the output of the output channel and the treatment position. In each case, the scanning magnets may be arranged in series.

In some implementations, there may be a single scanning magnet located within the beamline structure upstream of the output of output channel or elsewhere. For example, as shown in FIGS. 2 and 3, scanning magnet 30 may be located upstream of output channel 17 relative to the particle accelerator and at the input of output channel 17. Scanning magnet 30 may be configured to move the particle beam in two dimensions (for example, the Cartesian X and Y dimension). In this example, scanning magnet 30 may have the same construction and operation as the scanning magnet shown in FIG. 5. In this example, all particle beam movement is implemented by controlling current through one or more coils of the single scanning magnet.

In this regard, by positioning all or some of the scanning magnets within a beamline structure upstream of the nozzle, it may be possible to reduce the size of the particle therapy system relative to systems that implement scanning external to the gantry.

In some implementations, one or more the scanning magnets described herein may be superconducting. For example, one or more, including all, of the scanning magnets downstream of the output channel may be superconducting. For example, one or more, including all, of the scanning magnets within the beamline structure upstream of the nozzle may be superconducting. In this regard, it can be difficult to move the particle beam accurately in the presence of high magnetic fields such as those found in the beamline structure. Use of a superconducting magnet for scanning enables generation of magnetic fields of 2.5 T or greater or 3 T or greater to move the particle beam, which can overcome effects on the particle beam of the high magnetic fields, such as 2.5 T or greater or 3 T or greater, produced by the beamline structure.

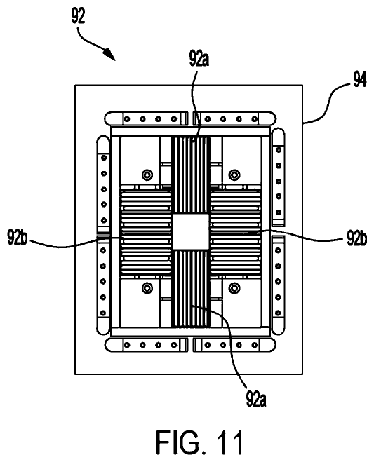

FIG. 11 shows an example implementation of a superconducting scanning magnet 92 configured to move the particle beam in two dimensions, which may be used in the scanning implementations described herein. In this example, scanning magnet 92 may have the same construction and operation as scanning magnet 43 shown of FIG. 5. Superconducting magnet 92 includes sets of high-temperature superconducting coils 92a and 92b, which are similar in construction to coils 46 and 45, respectively, of FIG. 5. Examples of high-temperature superconductors include, but are not limited to, YBCO (yttrium barium copper oxide) and BSCCO (bismuth strontium calcium copper oxide). Scanning magnet 92 is contained in a cryostat 94 that maintains the superconducting magnet at superconducting temperatures, e.g., above 77° Kelvin (K) or above 90° K. A cryostat may include a device configured to maintain the superconducting coils at cryogenic temperatures. The cryostat may maintain temperature by thermally isolating the superconducting coils from room temperature. This generally is performed using vacuum insulation, thermal radiation shields and/or superinsulation to reduce radiation heat transfer, and low thermal conductivity connections between room temperature and cryogenic temperatures. In some examples, liquid helium may be used to cool the coils to superconducting temperatures in the cryostat using, for example, conductive or immersive cooling. In conductive cooling, heat is transferred away from the superconducting coils using a thermal conductor. In immersive cooling, the superconducting coils may be in direct contact with a cryogen, such as liquid helium. In operation, current is applied to coils 92a and 92b to generate the magnetic fields used for scanning.

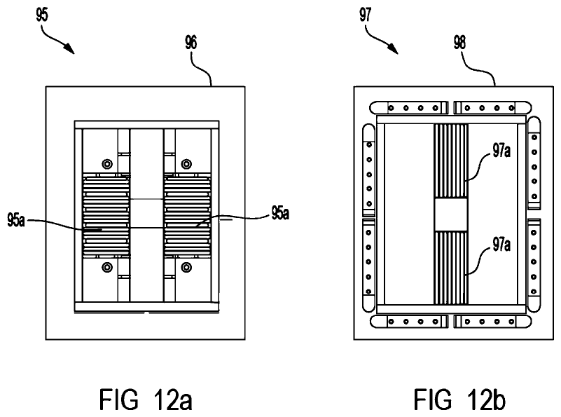

FIG. 12a shows an example of a superconducting magnet 95 configured to move the particle beam in one dimension only, which may be used in scanning implementations described herein. The superconducting magnet includes high-temperature superconducting coil set 95a, which is configured to move the particle beam one dimension only (for example, the Cartesian X or Y dimension). Examples of high-temperature superconductors include, but are not limited to, YBCO and BSCCO. Superconducting magnet 95 is contained in a cryostat 96 that maintains the superconducting magnet at superconducting temperatures, e.g., above 77° Kelvin (K). For example, liquid helium may be used to cool the coils to superconducting temperatures. Current is applied to coils 95a to generate the magnetic fields used for scanning. FIG. 12b shows an example of a superconducting scanning magnet 97 configured to move the particle beam in one dimension only. That dimension is different from, such as orthogonal to, the dimension that magnet 95 of FIG. 12a moves the particle beam. Superconducting magnet 97 includes high-temperature superconducting coil set 97a, which is configured to move the particle beam one dimension only (for example, the Cartesian X or Y dimension). Examples of high-temperature superconductors include, but are not limited to, YBCO and BSCCO. Superconducting magnet 95 is contained in a cryostat 98 that maintains the superconducting magnet at superconducting temperatures, e.g., above 77° Kelvin (K). For example, liquid helium may be used to cool the coils to superconducting temperatures. Current is applied to coils 97a to generate the magnetic fields used for scanning.

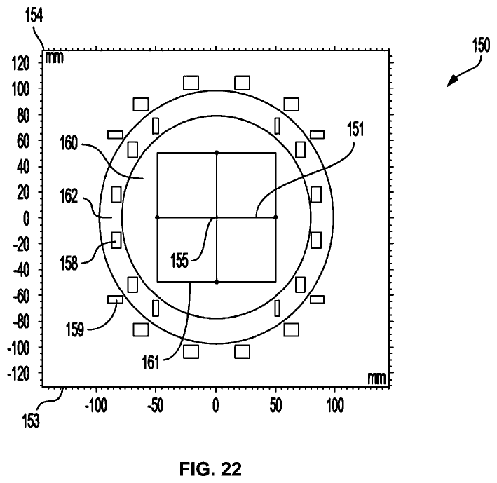

FIG. 22 shows a front, cut-away view of another example implementation of a superconducting scanning magnet 150 configured to move the particle beam in two dimensions, which may be used in the scanning implementations described herein. In this example, scanning magnet 150 may be contained in a cryostat (not shown) such as those described above to maintain the superconducting magnet at superconducting temperatures, e.g., between 30° K and 40° K in this example, although the cryostat is not limited to these temperatures. A cryocooler may be used to maintain the temperature of the cryostat at superconducting temperatures. A cryocooler includes a device for providing active cooling of the superconducting coils down to cryogenic temperatures. The cryocooler may be controlled by the control systems described herein.

In FIG. 22, grid 151 shows the scanning beam aperture in both the Cartesian X and Y dimensions, 153 and 154, respectively. For example, grid 151 shows that scanning magnet 150 can move the particle beam ±5 cm in the X dimension and ±5 cm in the Y dimension relative to a reference 0,0 point 155. In other implementations, the scanning magnet may be configured to move the particle beam over lengths that are more or less than ±5 cm in the X dimension and ±5 cm in the Y dimension. In FIG. 22, sets of superconducting coils 158 and 159 are wound around an electrically nonconductive or an electrically non-superconducting material 160 to create aperture 161 that contains grid 151. Inner superconducting coils 158 may be separated from outer superconducting coils 159 by an electrically nonconductive or an electrically non-superconducting material 162. Superconducting coils 158 may be configured so that the magnetic fields generated thereby are orthogonal to the magnetic fields generated by superconducting coils 159. And, superconducting coils 159 may be configured so that the magnetic fields generated thereby are orthogonal to the magnetic fields generated by superconducting coils 158. For example, the windings of superconducting coils 158 and 159 may be orthogonal to each other. In some implementations, the magnetic fields generated by superconducting coils 158 and 159 need not be orthogonal, but rather may be different—for example, at an angle to each other that is less than 90°—yet still enable scanning in a grid such as grid 151.

In this example, superconducting coils 158 control movement of the particle beam in the X dimension. For example, current runs through those superconducting coils to produce a magnetic field. The strength of that magnetic field is proportional to the amount of current running through the superconducting coils. And, the strength of the magnetic field is proportional to the amount that the particle beam moves in the X dimension during scanning. In this example, superconducting coils 159 control movement of the particle beam in the Y dimension. For example, current runs through those superconducting coils to produce a magnetic field. The strength of that magnetic field is proportional to the amount of current running through the superconducting coils. And, the strength of the magnetic field is proportional to the amount that the particle beam moves in the X dimension during scanning. Current may run through superconducting coils 158 and 159 at the same time to produce a cumulative magnetic field that moves the particle beam in both the X and Y dimensions. Current may run through superconducting coils 158 and 159 at different times so that the particle beam moves in the X or Y dimensions at separate times, but still reaches a target location.

An example of electrically non-superconducting material that may be included in scanning magnet 150 is copper; however, scanning magnet 150 is not limited to use with copper. The electrically non-superconducting material may promote heat dissipation, for example during a quench of the superconducting coils 158 and 159.

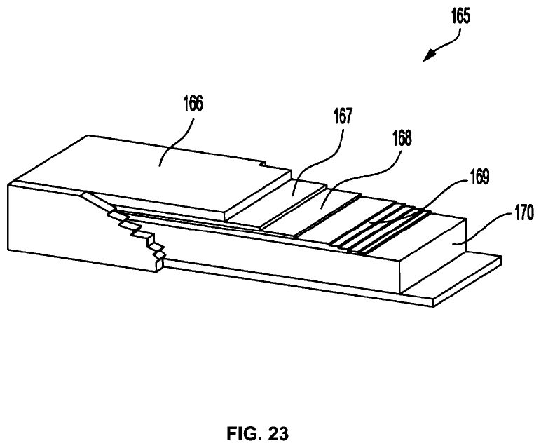

FIG. 23 shows a cross-section of high-temperature superconducting tape 165 that may be wound into coils to implement each of superconducting coils 158 and 159. Superconducting tape 165 includes a copper (Cu) stabilization layer 166 that encases or surrounds the other layers of superconducting tape 165. Superconducting tape 165 also includes a silver (Ag) cap layer 167, a rare-earth barium copper oxide (ReBCO) superconducting layer 168 (or layer(s) of other high-temperature superconducting material(s)) adjacent to and in contact with the silver cap layer, a buffer layer stack 169 adjacent to and in contact with the ReBCO superconducting layer to prevent interdiffusion between oxides and a metal substrate, and a substrate layer 170 adjacent to and in contact with the buffer layer stack. Examples of materials that may be included in the substrate layer include, but are not limited to, an electrically-conductive metal such as copper, nickel, or aluminum. Examples of materials that may be included in the buffer layer stack include, but are not limited to, SrRuO₃ (strontium ruthenate—SRO) and LaNiO₃ (LNO). Superconducting tape 165 may have a different configuration than that shown or may include different materials than those shown. For example, the copper stabilization layer may be omitted or a material other than copper may be used. Other types of superconducting materials may be used, such as YBCO and/or BSCCO.

Referring back to FIG. 3, output channel 17 portion of beamline structure 16 includes large-aperture superconducting magnetic dipole 31 arranged in series with large-aperture superconducting magnetic dipole 32. Examples of large apertures include, but are not limited to 10 cm by 10 cm, 20 cm by 20 cm, 30 cm by 30 cm, and so forth. Located between magnetic dipole 31 and magnetic dipole 32 are multiple large-aperture superconducting magnetic quadrupoles 33, 34, and 35. In this example, magnetic quadrupoles 33, 34, and 35 include, alternately, one or more focusing magnets and one or more defocusing magnets to focus and defocus the particle beam, respectively, in order to achieve a substantially consistent cross-sectional area of the particle beam. In this regard, the net effect on the particle passing through the alternating magnetic field gradients of the magnetic quadrupoles is to cause the beam to converge; that is, to focus. In some implementations, magnetic quadrupole 33 includes a defocusing magnet, magnetic quadrupole 34 includes a focusing magnet, and magnetic quadrupole 35 includes a defocusing magnet. In some implementations, magnetic 33 includes a focusing magnet, magnetic quadrupole 34 includes a defocusing magnet, and magnetic quadrupole 35 includes a focusing magnet. In some implementations, output channel 17 may include different numbers of magnetic quadrupoles in different configurations and/or a different number of magnetic dipoles in a different configuration. In some implementations, output channel 17 may include higher-order magnetics, such as sextupoles, in place of, or in addition to, the magnetic quadrupoles that are shown.

In some implementations, output channel 17 is configured to bend the particle beam in the presence of magnetic fields of 2.5 T, 3 T, or greater in the beamline structure. For example, the magnetic fields may be generated by running current through one or more coils in the magnets in the beamline structure, which may be on the order of 2.5 T or more, 3 T or more, 4 T or more, 5 T or more, 6 T or more, 7 T or more, 8 T or more, 9 T or more, 10 T or more, 11 T or more, 12 T or more, 13 T or more, 14 T or more, or 15 T or more. In the presence of magnetic fields such as these, the magnetics in output channel 17 are configured to produce a combined total bending angle of the particle beam anywhere in a range from 90° to 170°—for example, 90°, 95°, 100°, 105°, 110°, 115°, 120°, 125°, 130°, 135°, 140°, 145°, 150°, 155°, 160°, 165°, or 170°. Alternatively, in some implementations, output channel 17 is configured to bend the particle beam at a combined total bending angle that is less than 90° or that is greater than 170°—for example, 180° or greater. In FIGS. 1 to 3, output channel 17 is configured to bend the particle beam at a combined total bending angle of about 150° relative to line 38. To achieve a bending magnitude having a value from 110° to 170°, magnetic dipole 31 may be configured to bend the particle beam within a range of 20° to 85° relative to line 38, and magnetic dipole 32 may be configured to bend the particle beam within a range of 20° to 85° relative to horizontal line 38.

In some implementations, output channel 17 may include different numbers of magnetic structures in different configurations. For example, output channel 17 may include a magnetic dipole of the type described herein, followed by three alternating magnetic quadrupoles of the type described herein, followed by a magnetic dipole, followed by three alternating magnetic quadrupoles of the type described herein, followed by a magnetic dipole of the type described herein. Additional magnetics may be used, for example, to change where and by how much the particle beam bends. Additional magnetic structures may also be used to focus the particle beam over longer distances. Conversely, fewer numbers of magnetic structures may be used to focus the particle beam over shorter distances, as shown in FIG. 1 for example.

Nozzle 40 (FIG. 1) is connected to, and located at the output or exit of, the beamline structure output channel 17. The nozzle may be considered part of—for example, an extension of—the beamline structure, since particle beam from the output channel moves through the nozzle on its way to the treatment location. In the example of FIG. 1, nozzle 40 is a separate structure from output channel 17 and, where applicable, moves along with output channel. In some implementations, nozzle 40 may be an integral part of the output channel. Nozzle 40 is an example of a particle beam output device. In this example, nozzle 40 receives the particle beam from output channel 17 and, in some implementations, conditions the particle beam for output to an irradiation target, such as a tumor in a patient, at the treatment position or isocenter. In this regard, as noted, output channel 17 bends the particle beam by at least 90°. The particle beam is thus directed toward the treatment position or isocenter as it exits output channel 17. In addition, as described herein, scanning magnet(s) 30 may move the particle beam within a plane to move the particle beam across the irradiation target.

In this regard, as explained previously, the nozzle may contain one or more scanning magnets. The energy degrader is downstream of the scanning magnets and the collimator is downstream of the scanning magnets. In FIGS. 2 and 3, energy degrader 41 receives the scanning or moving particle beam from the scanning magnet(s). In this example, energy degrader 41 is mounted to gantry 14 (via nozzle 40) between output channel 17 and the irradiation target at treatment position 19. Energy degrader 41 is configured to, and controllable to, change an energy of the particle beam before the particle beam reaches the irradiation target. In some implementations, the energy degrader is the sole mechanism by which to actively control the change in energy of the particle beam prior to the particle beam reaching the irradiation target. In some implementations, the energy of the particle beam is not actively controllable after the particle beam is output by the particle accelerator and prior to the particle beam reaching the energy degrader. For example, in such implementations, components of the gantry between the particle accelerator and the energy degrader do not, and are not configured to, actively control the beam energy. Stated yet another way, the gantry or the beamline conduit thereof is not configured to actively control the particle beam energy after the particle beam is output by the particle accelerator and prior to the particle beam reaching the energy degrader. In some cases, there may be some incidental changes in energy caused by movement through the beamline structure; however, those changes are not actively controlled.

As noted previously, the particle beam output by the accelerator may be monoenergetic and the energy degrader is the only/sole or primary vehicle for changing beam energy during treatment of an irradiation target. An example monoenergetic particle beam includes a particle beam having a single, fixed energy level, such as 100 MeV, 150 Mev, 200 Mev, 250 Mev, and so forth. A monoenergetic particle beam may deviate from the fixed energy level by a predetermined amount, such as ±10%, ±5%, ±2%, or ±1%, and still be considered monoenergetic. Switching operation of the accelerator during treatment, as is required to switch particle beam energies during treatment, may produce excess stray neutrons, resulting in the need for increased shielding and reducing beamline efficiency. The neutrons may be generated by the particle accelerator and/or by magnetics along the beamline structure. By using a particle beam that is monoenergetic during treatment and relying on the energy degrader to change beam energy, production of stray neutrons may be reduced or minimized and the efficiency of the beamline structure may be increased.

In an example, the energy degrader may include plates that are movable into or out of a path of the particle beam. In another example, the energy degrader may include wedges that overlap at least in part and that are movable within a path of the particle beam. An example wedge is a polyhedron defined by two triangles and three trapezoidal faces. In either configuration, variable amounts of material are movable into the path of the particle beam. The material absorbs energy from the particle beam, resulting reduced-energy beam output. The more material there is in the path of the particle beam, the less energy that the particle beam will have. In some implementations, the energy-absorbing structures are movable across all of the beam field or across only part of the beam field. As noted, in some examples, the beam field includes the maximum extent that the particle beam can be moved across a plane parallel to the treatment area on a patient for a given position of the compact gantry.

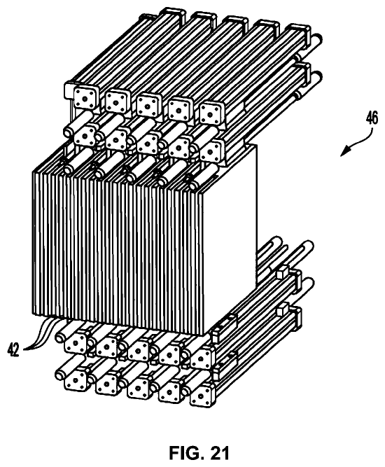

Referring to FIG. 21, in an example, energy degrader 46 is a range modulator that is controllable to move structures 42 into, and out of, the path of the particle beam to change the energy of the particle beam and therefore the depth to which dose of the particle beam will be deposited in the irradiation target. Examples of such energy-absorbing structures include, but are not limited to, plates; polyhedra such as wedges, tetrahedra, or toroidal polyhedra; and curved three-dimensional shapes, such as cylinders, spheres, or cones. In this way, the energy degrader can cause the particle beam to deposit doses of radiation in the interior of an irradiation target to treat layers or columns of the target. In this regard, when protons at a particular energy move through tissue, the protons ionize atoms of the tissue and deposit a dose primarily at a predefined tissue depth corresponding to that energy. The energy degrader thus is configured to move the particle beam in the Cartesian Z dimension through the target, thereby enabling the scanning magnet to perform scanning in a third dimension (Cartesian Z) in addition the Cartesian X and Y dimensions. In some implementations, an energy absorbing structure of the energy degrader, such as a plate or wedge, may be configured to move during movement (scanning) of the particle beam and track or trail the particle beam during movement. An example energy degrader that tracks or trails particle beam movement is described in U.S. Pat. No. 10,675,487 (Zwart) entitled “High-Speed Energy Switching”. The content of U.S. Pat. No. 10,675,487, particularly the content related to the energy degrader that tracks or trails particle beam movement (e.g., FIGS. 36 to 46 of U.S. Pat. No. 10,675,487 and the accompanying description), is incorporated herein by reference.

The Bragg peak is a pronounced peak on the Bragg curve that plots the energy loss of ionizing radiation during travel through tissue. The Bragg peak represents the depth at which most radiation deposits within tissue. For protons, the Bragg peak occurs right before the particles come to rest. Accordingly, the energy of the particle beam may be changed to change the location of its Bragg peak and, therefore, where a majority of the dose of protons will deposit in depth in the tissue. In this regard, the particle accelerator may be a fixed-energy particle accelerator. In a fixed-energy particle accelerator, the particle beam always exits the particle accelerator at the same, or about the same, energy—for example, within a 10%, 5%, or 1% deviation or less from an expected or target energy. In a fixed-energy particle accelerator, the energy degrader is the primary vehicle or the sole vehicle for varying the energy of the beam applied to an irradiation target in the patient. In some implementations, the particle accelerators described herein are configured to output particle beams at a single energy or at two or more energies within a range between about 100 MeV and about 300 MeV (for example, between 115 MeV and 250 MeV). The fixed energy output may be within that range (e.g., 250 MeV) or, in some examples, above or below that range.

In some implementations, the particle accelerator is a dual-energy accelerator. In a dual-energy particle accelerator, the particle beam exits the particle accelerator at one of two different energy levels—a high energy level or a low energy level. The terms “high” and “low” have no specific numerical connotations but rather are intended to convey relative magnitudes. In some implementations, the particle accelerators described herein are configured to output particle beams at two energies that are within a range that is between about 100 MeV and about 300 MeV. The high energy output and the low energy output may be values within that range or, in some examples, above or below that range. The energy degrader described herein may be used with dual-energy particle accelerators in order to reduce the energy of the particle beam below one of the two energy levels and/or to finely adjust between the two energy levels.

In the figures, nozzle 40 also includes a collimator 44 downstream of energy degrader 41 relative to the particle accelerator (that is, closer to the irradiation target). In an example, a collimator is a structure that is controllable to allow some radiation to pass to a target and to block some radiation from passing to the patient. Typically, the radiation that passes is directed to an irradiation target to be treated, and the radiation that is blocked would otherwise hit, and potentially damage, healthy patient tissue. In operation, the collimator is placed in the radiation path between output channel 17 and the irradiation target and is controlled to produce an opening of an appropriate size and shape to allow some radiation to pass through the opening to the irradiation target, while a remainder of the structure blocks some radiation from reaching adjacent tissue.

The collimator may be configurable—for example, its aperture may be controlled and changed during treatment. The collimator may be fixed or not changeable. For example, the collimator may have a fixed shape that cannot be altered.

In some implementations, components of an example configurable collimator include multiple leaves that are dynamically reconfigurable during movement of the particle beam to change a shape of an edge defined by the multiple leaves. The edge is movable between at least a portion of the particle beam and a target of the particle beam so that a first part of the particle beam on a first side of the edge is at least partly blocked by the multiple leaves and so that a second part of the particle beam on a second side of the edge is allowed to pass to the target.

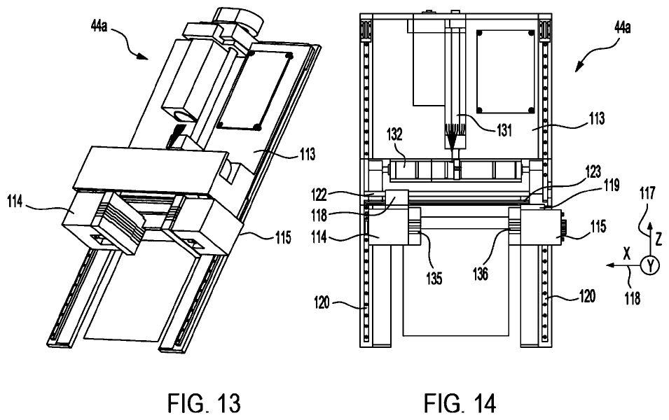

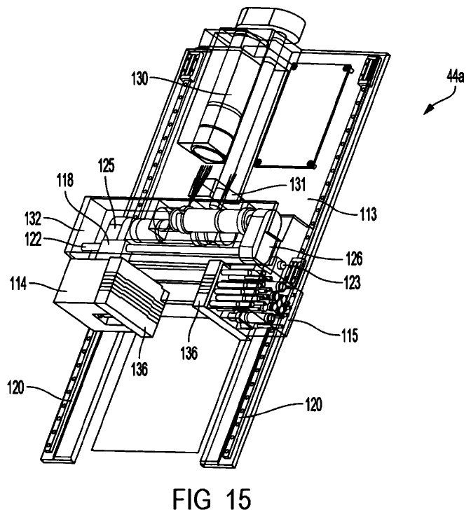

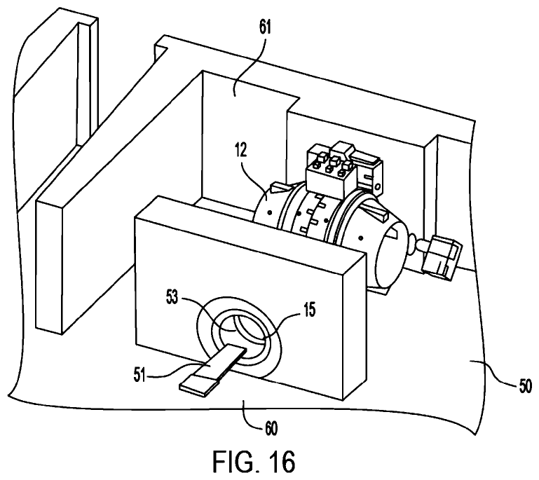

FIGS. 13, 14, and 15 show an example implementation of configurable collimator 44a, which may be used with the particle therapy system described herein. Collimator 44a including carriages 113, 114, and 115 configured to hold, and to move, the leaves described above both vertically and horizontally relative to an irradiation target. As shown, vertical movement includes movement in the Cartesian Z-dimension 117, and horizontal movement includes movement in the Cartesian X dimension 118 (with the Cartesian Y dimension being into, or out of, the page in FIGS. 13 and 14). FIGS. 14 and 15 show parts of carriage housings as transparent in order to show components inside the housings; however, the housings are not actually transparent.

Carriage 113 is referred to herein as the primary carriage, and carriages 114 and 115 are referred to herein as secondary carriages. Secondary carriages 114, 115 are coupled to primary carriage 113, as shown in FIGS. 13 to 15. In this example, secondary carriages 114, 115 each include a housing that is fixed to primary carriage 115 via a corresponding member 118, 119. In this example, primary carriage 113 is movable vertically (the Z dimension) relative to the irradiation target and relative to particle accelerator along tracks 120. The vertical movement of primary carriage 113 also causes the secondary carriages to move vertically. In some implementations, the secondary carriages move vertically in concert.

As shown in FIGS. 13 to 15, each secondary carriage 114, 115 is connected to a corresponding rod or rail 122, 123, along which the secondary carriage moves. More specifically, in this example, motor 125 drives secondary carriage 114 to move along rod 122 toward or away from secondary carriage 115. Likewise, in this example, motor 126 drives secondary carriage 115 to move along rod 123 toward or away from secondary carriage 114. Control over movement of the primary and secondary carriages is implemented to position the leaves relative to the irradiation target, as described herein. In addition, the leaves themselves are also configured to move in and out of the carriages, as also described herein.

As shown in FIG. 15, a motor 130 drives the vertical movement of primary carriage 113. For example, as shown in FIG. 15, lead screw 131 is coupled to housing 132, which holds motors 125, 126 that drive corresponding secondary carriages 114, 115, and which is mounted on tracks 120. Lead screw 131 is coupled to, and driven vertically by, motor 130. That is, motor 130 drives lead screw 131 vertically (the Cartesian Z dimension). Because lead screw 131 is fixed to housing 132, this movement also causes housing 132, and thus secondary carriages 114, 115, to move along tracks 120, either toward or away from the irradiation target.

In this example implementation, seven leaves 135, 136 are mounted on each secondary carriage 114, 115. Each secondary carriage may be configured to move its leaves horizontally into, or out of, the treatment area. Using linear motors, the individual leaves on each secondary carriage may be independently and linearly movable in the X dimension relative to other leaves on the same secondary carriage. In some implementations, the leaves may also be configured to move in the Y dimension. Furthermore, the leaves on one secondary carriage 114 may be movable independently of the leaves on the other secondary carriage 115. These independent movements of leaves on the secondary carriages, together with the vertical movements enabled by the primary carriage, allow the leaves to be moved into various configurations. As a result, the leaves can conform, both horizontally and vertically, to treatment areas that are randomly shaped both in horizontal and vertical dimensions. The sizes and shapes of the leaves may be varied to create different conformations. For example, the sizes and shapes may be varied to treat a single beam spot and, thus, a single column. In some implementations individual leaves on each secondary carriage may be independently and linearly movable using electric motors that drive lead screws in the X dimension relative to other leaves on the same secondary carriage.

The leaves may be made of any appropriate material that prevents or inhibits transmission of radiation. The type of radiation used may dictate what material(s) are used in the leaves. For example, if the radiation is X-ray, the leaves may be made of lead. In the examples described herein, the radiation is a proton or ion beam. Accordingly, different types of metals or other materials may be used for the leaves. For example, the leaves may be made of nickel, tungsten, lead, brass, steel, iron, or any appropriate combinations thereof. The height of each leaf may determine how well that leaf inhibits transmission of radiation.

Implementations of the configurable collimator described with respect to FIGS. 13 to 15 are described in U.S. Patent Publication No. 2017/0128746 (Zwart) entitled “Adaptive Aperture”. The content of U.S. Patent Publication No. 2017/0128746, particularly the content relating to the description of the adaptive aperture (e.g., FIGS. 1 to 7 of U.S. Patent Publication No. 2017/0128746 and the accompanying description), is incorporated herein by reference.

Referring back to FIG. 1, as noted, example particle therapy system includes an isocentric gantry that is compact in size, which reduces overall system size. In implementations of compact gantry 14, the diameter of support structure 15 may be less than 6 meters (m), less than 5 m, or less than 4 m. In an example, the diameter of support structure 15 is 4.8 m. The length of the beamline structure may be measured from, and equal to the distance between, the output of the accelerator and the system isocenter. In implementations of compact gantry 14, the length of beamline structure 16 may be less than 6 meters (m), less than 5 m, less than 4.5 m, or less than 4 m. In an example, the length of beamline structure 16 is 4.2 m. In this regard, the distance between the particle accelerator and the system isocenter or treatment position may be less than 6 m, less than 5 m, less than 4.5 m, or less than 4 m. In implementations of compact gantry 14, the distance between the output of output channel 17 and the system isocenter or the treatment position is 2 m or less, 1.5 m or less, or 1 m or less. In implementations of compact gantry 14, the distance between the output of output channel 17 and the system isocenter or the treatment position is between 0.8 m and 1.4 m. In an example, the distance between the output of output channel 17 and the system isocenter or the treatment position is 1.01 m. Other implementations may have different dimensions than those listed here.

In some implementations, the particle therapy system has a footprint of 93 square meters (m²) or less or of 75 m² or less. In some implementations, the particle therapy system is configured to fit within a vault designed for a LINAC. For example, the components of FIGS. 1 to 3 may be small enough fit within, and have dimensions that fit within, a vault having the following dimensions: 25 feet (7.62 m) or less in length, 20 feet (6.09 m) or less in width, and 11 feet (3.35 m) or less in height. For example, the components of FIGS. 1 to 3 may be small enough fit within, and have dimensions that fit within, a vault having the following dimensions: 25 feet (7.62 m) or less in length, 26 feet (7.92 m) or less in width, and 10 feet (3.05 m) or less in height. For example, the components of FIGS. 1 to 3 may be small enough fit within, and have dimensions that fit within, a LINAC vault having a footprint of 26.09 feet (11 m) or less by 29.62 feet (9 m) or less, with a height of 16.40 feet (5 m) or less. However, as noted, some implementations of the particle therapy system may have different dimensions including, but not limited to, diameters, heights, widths, and lengths. In some implementations, the ceiling of a pre-existing LINAC vault may not be high enough to support full 360° rotation of or around the gantry. In such implementations, a pit 90 (FIG. 1) may be dug beneath the floor of the LINAC vault to enable the rotation.

FIGS. 1 and 16 shows examples of treatment spaces 49 and 50 in which particle therapy system 10 and its variants may be housed. The treatment spaces are implemented in LINAC vaults in these examples, which may be shielded using lead or other appropriate materials such as concrete, borated polyethylene, and/or steel. In this regard, particles, such as protons, that are created by the particle accelerator but do not reach the irradiation target create secondary radiation through the production of high energy neutrons. In an example, particle accelerator 12 and/or gantry generates 10 millisieverts or less of such neutrons per gray of dose delivered by the particle beam.

Use of a monoenergetic particle bean and reliance on an energy degrader that is outside of the magnetics in the beamline structure enables the magnetics in the beamline to direct the beam efficiently. More specifically, changes in beam energy within the beamline increase production of stray neutrons and, therefore, losses of particle beam within the beamline, thereby degrading its efficiency. The monoenergetic particle beam used in the implementations of the systems described herein, combined with the magnetic structures in the beamline, may lead to increased efficiency. In some cases, decreases in the length of the beamline structure may also increase efficiency. In some implementations, the variants of the beamline structure described herein have an efficiency of 10% or more, 20% or more, 30% or more, 40% or more, 50% or more, 60% or more, 70% or more, 80% or more, or 90% or more. In some examples, efficiency is a measure of the percentage of particles output from the particle accelerator that are output from the beamline structure. So, an efficiency of 10% or more includes 10% or more of the particles output from the particle accelerator being output from the beamline structure; an efficiency of 20% or more includes 20% or more of the particles output from the particle accelerator being output from the beamline structure; an efficiency of 30% or more includes 30% or more of the particles output from the particle accelerator being output from the beamline structure; an efficiency of 40% or more includes 40% or more of the particles output from the particle accelerator being output from the beamline structure; an efficiency of 50% or more includes 50% or more of the particles output from the particle accelerator being output from the beamline structure; an efficiency of 60% or more includes 60% or more of the particles output from the particle accelerator being output from the beamline structure; an efficiency of 70% or more includes 70% or more of the particles output from the particle accelerator being output from the beamline structure; an efficiency of 80% or more includes 80% or more of the particles output from the particle accelerator being output from the beamline structure; and an efficiency of 90% or more includes 90% or more of the particles output from the particle accelerator being output from the beamline structure. In an example, the particle accelerator and gantry described herein transmit more than 70% of a proton beam to a patient even at energies in lower range of the accelerator.

Beamline efficiency of the type described herein enables a “single room” solution in which the particle accelerator, the gantry, and patient all reside with a single vault, as described above. Within this vault, the particle accelerator itself may include shielding, but separate compartments 60 and 61 (see FIG. 16) in the vault containing the patient and the particle accelerator, respectively, need not be shielded from each other. In other words, in some implementations, there is no electromagnetic shielding that is external to the particle accelerator and the gantry that separates the particle accelerator from the patient. Shielding may not be needed due to the low levels of neutrons emitted by the system. In some implementations, there may be minimal shielding between the separate compartments 60 and 61. For example, the shielding may be 30 cm or less in thickness, 20 cm or less in thickness, or 10 cm or less in thickness.

Referring also to FIG. 1, particle therapy system 10 also includes a treatment couch 51. Treatment couch 51 is configured to move relative to hole 53 in or through gantry 14 to position a patient at the system isocenter or treatment position. In this example, treatment couch 51 is mounted to a robotic arm 54. Arm 54 includes a first segment 55, a second segment 56, and third segment 57. First segment 55 is rotatably coupled to second segment 56 and second segment 56 is rotatably coupled to third segment 57. Treatment couch 51 is coupled to third segment 57 as shown in the figure. Arm 54 is controllable to move treatment couch 51 in and through hole 53 to position a patient lying on the couch for treatment; that is, to move the patient into the treatment position. In some implementations, arm 54 may position the patient in two degrees of freedom, in three degrees of freedom, in four degrees of freedom, in five degrees of freedom, or in six degrees of freedom. An example of two degrees of freedom is forward-backward movement and left-right movement; an example of three degrees of freedom is forward-backward movement, left-right movement, and up-down movement; an example of four degrees of freedom is forward-backward movement, left-right movement, up-down movement and one of pitch, yaw, or roll movement; an example of five degrees of freedom is forward-backward movement, left-right movement, up-down movement and two of pitch, yaw, or roll movement; and an example of six degrees of freedom is forward-backward movement, left-right movement, up-down movement, pitch movement, yaw movement, and roll movement. In some implementations, the treatment couch may be replaced by or include a couch that inclines at least in part or that is convertible to a chair, and that is still be controllable in two, three, four, five, or six degrees of freedom to position the patient for treatment. In some implementations, arm 54 may have a different configuration than that shown in FIG. 1. For example, arm 54 may have two segments or more than three segments. Hydraulics, robotics, or both, may control or implement non-planar movement of the treatment couch.

In some implementations, output channel 17 may rotate at least part-way, including all the way, around support structure 15 or output channel may remain fixed on support structure 15 and all or part of support structure 15 may rotate around the treatment position. In some implementations, output channel 17 may not rotate around support structure 15 and the support structure may not rotate around the patient. Instead, the output channel may remain stationary, thereby providing a particle beam that is fixed in one direction. In implementations such as these, the treatment couch or other seat moves relative to the fixed beam during treatment. In some system described herein, the location of the particle beam may be set through rotation of the gantry, after which the beam remains fixed except for scanning movements across the irradiation target and the treatment couch or other seat moves during treatment. In some implementations, treatment may be implemented using a combination of gantry movement and treatment couch (or other seat movement). For example, the output channel may be positioned and the beam may be fixed temporarily, during which time the treatment couch moves to implement treatment. After that, the output channel may be repositioned to fix the beam temporarily at a new position. Treatment may be implemented at the new position through couch movement. These operations may be repeated as defined by a treatment plan drafted for use with the particle therapy system.

Particle therapy system 10 may be an intensity-modulated proton therapy (IMPT) system. IMPT systems enable spatial control of circumscribed beams of protons that may have a variable energy and/or intensity. IMPT takes advantage of the charged-particle Bragg peak—as noted, the characteristic peak of dose at the end of particles' delivery range—combined with the modulation of particle beam variables to create target-local modulations in dose that achieve objectives set forth in a treatment plan. IMPT may involve directing particle beams toward the irradiation target at different angles and at different intensities to treat the target. In some implementations, the particle beam may be scanned—for example, moved—across layers of the irradiation target, with each layer being treated one or more times from the same or different angles. Movement across the irradiation target to implement scanning may be performed using the scanning magnet(s) described herein,

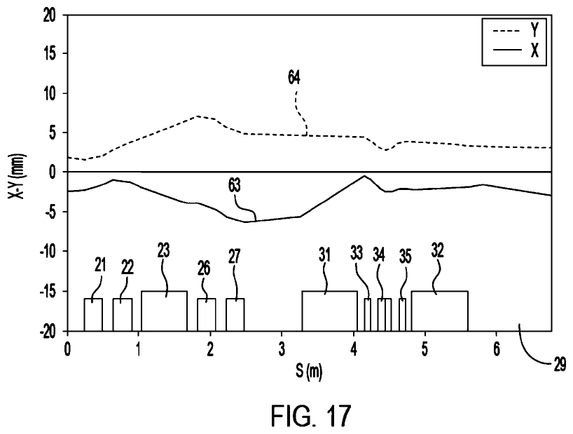

FIG. 17 shows example horizontal (x) beam envelope 63 and vertical (y) beam envelope 64 (e.g., cross-sections) along the length 29 the compact gantry described herein. The x and y dimensions of the beam spot cross-section are determined for magnetic quadrupoles 21 and 22, magnetic dipole 23, magnetic quadrupoles 26 and 27, magnetic dipole 31, magnetic quadrupoles 33, 34, and 35, and magnetic dipole 32. Beam sizes are determined based on calculations of beam optics using measured beam parameters at the exit of particle accelerator 12 and the design parameters of all the beamline magnets. In some implementations, the beam spot radius at the isocenter (e.g., a treatment positions) is approximately 3 millimeters (mm) for both x and y. In some implementations, the beam spot radius at the isocenter (e.g., a treatment positions) is approximately 4 mm for both x and y. In some implementations, for 200 MeV to 230 MeV proton beams, magnetic fields at the magnetic dipoles in beamline structure 16 are no more than 4 T and the bending radius of the beam at each of the magnetic dipoles is approximately 0.6 meters. In some implementations, for 200 MeV to 230 MeV proton beams, magnetic fields at the magnetic dipoles in beamline structure 16 are at least 3 T, that is, 3 T or greater. As noted, the systems described herein are not limited to these parameter values and some implementations may have different dimensions, energies, and magnetic fields.

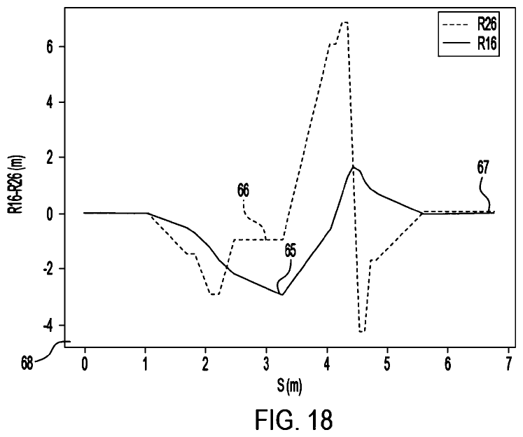

Chromatic-aberration correction can occur in a beamline having dispersion, generated by inclusion of dipole magnets and multiple correctors in dispersive regions. The standard definition for an achromat is a beam transport line having zero values for spatial dispersion (R16) and angular dispersion (R26). Referring to FIG. 18, the magnetics in implementations of the compact gantry may be configured to be achromat—e.g., both R16 65 and R26 66 of the beam transfer matrix elements equal zero at the isocenter, which is at or near 0 m along the beamline structure length 68 (the X-axis). Reducing or minimizing spatial and angular beam dispersions may be consequential to pencil beam scanning techniques implemented by the particle therapy systems described herein. In this regard, in some pencil beam scanning techniques, the cross-section of the particle beam is required to be substantially round at the isocenter. As such, the beam spot size in both x and y (FIG. 18) planes should be close at the isocenter 67. During beam scanning, changes to the beam shape and beam diameter over the entire scanning area should be reduced or minimized, otherwise, different beam particles of different energies may land at different locations in the bending plane. This may cause the beam shape and beam size to differ in another plane.

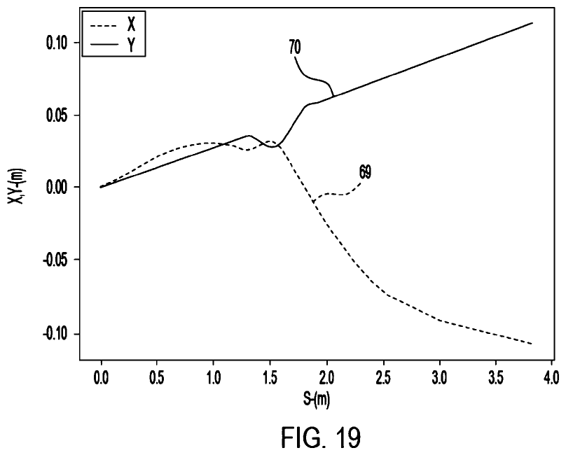

FIG. 19 shows examples of beam scans in the x dimension 69 and the y dimension 70. The firing of the scanning magnets allows the beam particle to be deflected to an angle proportional to the field strength of the scanning magnets. In the example of FIG. 19, a beam scanning range that fully covers a beam field area of 20 cm by 20 cm is shown with beam deflection angles of approximately ±20 milliradians (mrad) and ±30 mrad from the scanning magnets. In this example, the source-to-isocenter distance (SAD) (that is, the accelerator to isocenter distance) is approximately 4 meters. In some implementations, from the scanning magnet(s) to the exit of output channel 17, the beam bending angle can be as large as 110° to 170°.

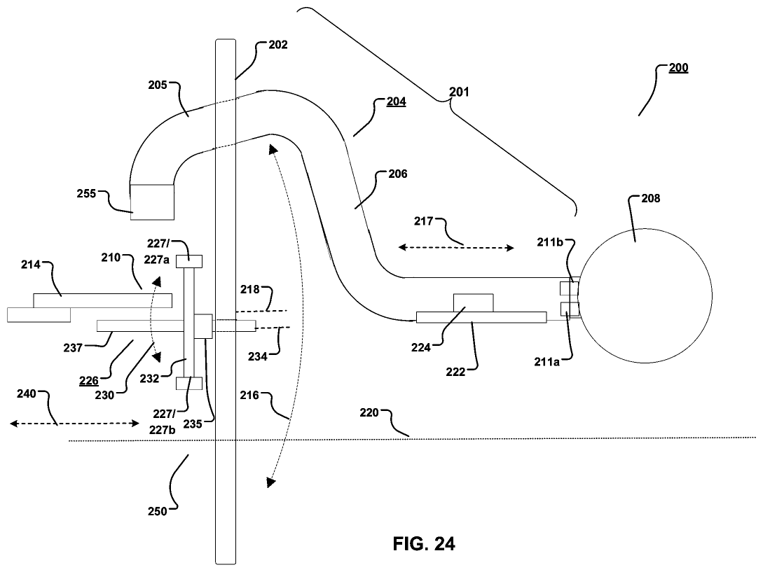

FIG. 24 shows an example of a particle therapy system 200 containing a gantry 201. Particle therapy system 200 and/or gantry 201 may include any or of the features described herein with respect to FIGS. 1 to 23. For example, gantry 201 may be, or include all or some of the features of, the gantries described with respect to FIGS. 1 to 4, 6, 9, 10 and the variants thereof described herein. In this regard, as described with respect to FIG. 1, gantry 201 includes ring-shaped or circular support structure 202 and a beamline structure 204 Beamline structure 204 includes an output channel 205 that mounts to support structure 202, a conduit 206 that directs the particle beam to the output channel, and a nozzle 255 that conditions the particle beam. As noted, the nozzle may be considered part of—for example, an extension of—the beamline structure and is referred to as a separate element only for the sake of illustration.

Gantry 201 connects mechanically to a particle accelerator 208, thereby enabling a particle beam to pass from particle accelerator 208 through gantry 201 to a target at a treatment position 210. Particle accelerator 208 may be any of the particle accelerators described herein, such as particle accelerator 12 of FIG. 1. In some implementations, gantry 201 may be configured to connect to, and to disconnect from, the particle accelerator. For example, mechanical connectors 211a on the gantry may mate to counterpart connectors 211b on the particle accelerator. The connectors may be push-pull connectors that lock. For example, the connectors may engage and disengage using applied axial force. The resulting connection created may be air-tight.

Beamline structure 204 may be configured to move rotationally around circular support structure 202, and thus around the treatment position 210 at couch 214. This rotational movement is represented by arrows 216. Beamline structure 204 is also configured to move translationally in the forward and backward directions of arrows 217 relative to treatment position 210. This type of translational movement can be characterized as being along a longitudinal dimension of the beamline structure, along the beamline, or along the axis of rotation around circular support structure 202—that is, parallel to an axis 218 that passes through a center of circular support structure 202. In FIG. 24, part of circular support structure 202 is below floor 220 and there is a cut-out (not shown) in the floor, through which at least part of beamline structure 204 passes during rotation around circular support structure 202.

To implement translational movement, beamline structure 204 may be held on a mount 222. In an example, mount 222 may include one or more tracks or rails, as shown in FIG. 24. Beamline structure 204 may be physically connected to the rails to enable movement of the beamline structure. In some implementations, the beamline structure may move along the rails. In some implementations, the rails themselves may move, thereby implementing movement of the beamline structure. Support structure 202 may include a collection of rollers or a rail at points of the intersection between the beamline structure and the support structure to enable the beamline structure to move translationally relative to, and/or through the support structure.

A motor 224 controls the movement of beamline structure 204 to move toward treatment position 210 and away from particle accelerator 208, and to move back away from treatment position 210 and toward particle accelerator 208. Although one motor 224 is shown, multiple motors may be used to implement the movement or along mount 222. Motor 224 may control movement of beamline structure 204 along a rail or, as noted, beamline structure 204 may be fixed to the rail and the motor may control movement of the rail and thereby control movement of the beamline structure. A control system, such as those described herein, may control operation of motor 224.

Particle therapy system 200 also includes mount 226, which is configured to hold an imaging system 227 comprised of one or more imaging devices 227a and 227b (examples of which are described below), and configured to enable rotational movement of the imaging devices relative to an irradiation target at the treatment position. Although only two imaging devices are shown in FIG. 24, any appropriate number of imaging devices may be used. The rotational movement is represented by arrows 230 in FIG. 24. The second mount 226 may include ring-shaped or circular support structure 232 having a central axis 234 around which the imaging devices are configured to rotate. A motor 235 may implement the rotation. The second mount may also include a track or rail 237 at or near central axis 234. Circular support structure 232 may be mechanically connected to the rail 237 to enable translational movement of support structure 232, and thus translational movement of imaging system 227, along central axis 234 relative to treatment position 210. This translational movement is represented by arrows 240. Motor 235 controls the movement of support structure 232 to move toward treatment position 210 and away from particle accelerator 208, and to move back away from treatment position 210 and toward particle accelerator 208.

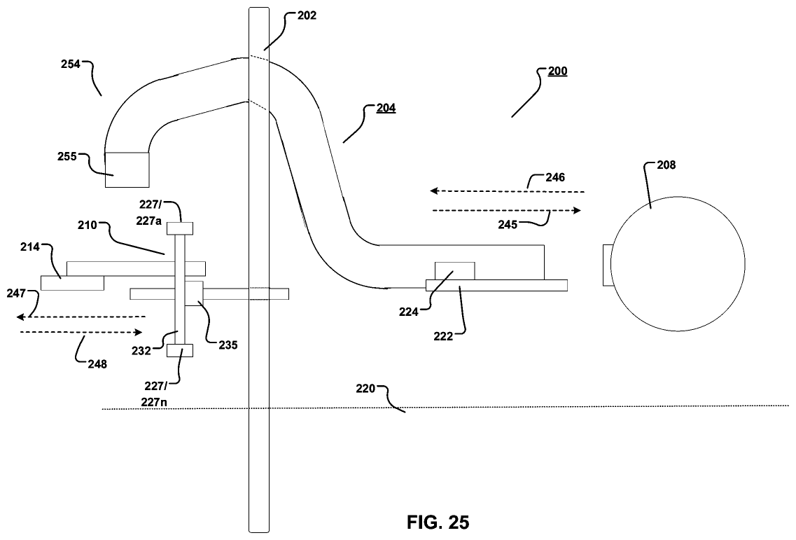

In the example of FIGS. 24 and 25 (connectors 211a, 211b omitted from FIG. 25), the entirety of beamline structure 204—which is the part of gantry 201 configured for translational movement in this example—is controllable to move in the directions of arrows 217. Nozzle 255, which is connected to and considered part of the beamline structure 204, moves along with the rest of beamline structure 204. To implement the movement, a control system—which may include any of the control system features described herein—may instruct motor 224 to move beamline structure 204 away from treatment position 210 and the amount of that movement. In some implementations, the translational movement of beamline structure 204 away from treatment position 210 in the direction of arrow 246 and later back toward treatment position 210 in the direction of arrow 245 (described below), is for at least 30 cm, for between 30 cm and 1 m, or for more than 1 m. In general, any appropriate amount of translational movement may be implemented. To implement movement in the direction of arrow 246, the motor generates enough force to cause connectors 211a (shown only in FIG. 24) on gantry 201 to disengage from their counterpart connectors 211b (shown only in FIG. 24) on particle accelerator 208.

In the example of FIG. 25, the entirety of support structure 232 and thus imaging system 227 is controlled to move in the direction of arrow 247 toward treatment position 210. To implement the movement, the control system may instruct motor 235 to move support structure 232 toward treatment position 210 and the amount of that movement. In some implementations, the translational movement of support structure toward treatment position 210 in the direction of arrow 247, and later back toward the accelerator in the direction of arrow 248 (described below), is for at least 30 cm, for between 30 cm and 1 m, or more than 1 m. In general, any appropriate amount of translational movement may be implemented.

In the example of FIGS. 24 and 25, the control system controls beamline structure 204 to move away from a region proximate to—for example, above—treatment position 210 and controls imaging system 227 to move into that region—for example, above treatment position 210 (FIG. 25). This is done in order to move the gantry out of the way of the imaging system so that the imaging system can capture images of the irradiation target in a patient at the treatment position. Furthermore, during movement of beamline structure 204, during movement of imaging system 227, and during image capture operations performed by the imaging system, couch 214 is controlled by the control system not to move, that is, to remain stationary. The patient, to the extent possible, is also controlled physically during this time to remain stationary on couch. The captured images are sent to the control system for analysis. The analysis result may be used to generate a treatment plan and/or to modify an existing treatment plan.

Following image capture, in the configuration of FIG. 25, the entirety of support structure 232 and thus imaging system 227 is controlled to move in the direction of arrow 248, toward accelerator 208. This movement results in repositioning imaging system 227 to its original position 250 shown in FIG. 24. To implement the movement, the control system may instruct motor 235 to move support structure 232 toward accelerator 208 and the amount of that movement. As was the case above, in some implementations, the translational movement of support structure 232 toward the accelerator in the direction of arrow 248 is for at least 30 cm, for between 30 cm and 1 m, or for more than 1 m. In general, the amount of movement in the direction of arrow 248 should be equal and opposite to the amount of movement in the direction of arrow 247.

Also following image capture, the beamline structure 204 is controlled to move in the direction of arrow 245 to reconnect to accelerator 208. This movement results in repositioning beamline structure 204 to the position shown in FIG. 24. To implement the movement, the control system may instruct motor 224 to move beamline structure 204 toward treatment position 210 and the amount of that movement. In general, the amount of movement in the direction of arrow 245 should be equal and opposite to the amount of movement in the direction of arrow 246. To implement movement in the direction of arrow 245, motor 224 generates enough force to cause connectors 211a (FIG. 24) on gantry 201 to reengage their counterpart connectors 211b (FIG. 24) on particle accelerator 208. In some implementations, the control system need not instruct the amount of movement back toward the accelerator, but rather may monitor the connectors and stop movement when the connectors have reengaged.

During the operations described with respect to FIGS. 24 and 25, the imaging system and the beamline structure may move concurrently or in sequence. Referring to FIG. 25, in an example sequential movement, beamline structure 204 may move away from the treatment position 210 in the direction of arrow 246 to position 254. Then, support structure 232 may move the imaging system from its original position 250 (FIG. 24) so that it aligns with treatment position 210 in the direction of arrow 247. Then, after the imaging system performs image capture operations, support structure 232 may move the imaging system 227 away from treatment position 210 in the direction of arrow 248 back to its original position 250 shown in FIG. 24 Then, beamline structure 204 may move in the direction of arrow 245 so that it aligns to treatment position 210 and connects to the particle accelerator, as shown in FIG. 24.

In a concurrent movement scenario, beamline structure 204 moves in the direction of arrow 246 to position 254 (FIG. 25) at the same time as, or during part of the same time as, support structure 232 moves imaging system 227 in the direction of arrow 247 toward treatment position 210. Following image capture by the imaging system, beamline structure 204 moves in the direction of arrow 245 toward treatment position 210 at the same time as, or during part of the same time as, support structure 232 moves imaging system 227 in the direction of arrow 248 toward its original position 250. Beamline structure 204 and support structure 232 may move at the same speed as support structure 232 or their speeds may be controlled to ensure that the beamline structure and the support structure do not collide during movement.

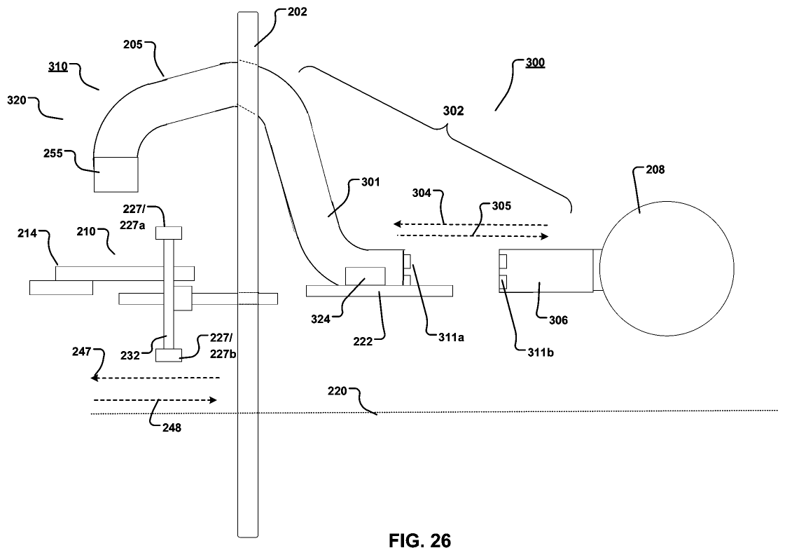

In the examples of FIGS. 26 to 34 below, the components shown and described may have substantially the same structure and function as components shown and described with respect to FIGS. 24 and 25, unless otherwise explained. In this regard, the same reference numerals in different figures indicate the same components.

In particle therapy system 300 of FIG. 26, only part 301 of beamline structure 302 is controllable to move translationally in the directions of arrows 304 and 305, while the remainder 306 of beamline structure 302 remains connected to particle accelerator 208. In this configuration, the amount of gantry 310 that moves is less than, and thus lighter than, the amount that moves in the configuration shown in FIG. 25. As a result, a smaller motor 324 may be used and less energy may be expended. In some implementations, part 301 may be 1 m, 2 m, 3 m, or less in length along the beamline for an example 4.2 m beamline structure. Generally, however, part 301 may have any appropriate length. In this example, connectors 311a, 311b may be the same as connectors 211a, 211b. However, in this case, mating connectors 311b are on part 306 of the gantry that remains connected to accelerator 208, and not on the accelerator itself. The operation of those connectors is described with respect to FIGS. 24 and 25.

To implement translational movement of gantry part 301 the control system may instruct motor 324 to move gantry part 301 away from treatment position 210 and the amount of that movement. In some implementations, the translational movement of gantry part 301 away from the accelerator in the direction of arrow 304, and later back toward the accelerator in the direction of arrow 305, is for at least 30 cm, for between 30 cm and 1 m, or more than 1 m. In general, any appropriate amount of translational movement may be implemented. To implement movement in the direction of arrow 304, the motor generates enough force to cause connectors 311a on gantry part 301 to disengage from their counterpart connectors 311b on gantry part 306.

Control over movement of support structure 202 and thus imaging system 227 in the direction of arrow 247 shown in FIG. 26 is as described with respect to FIGS. 24 and 25. As described above, during movement of gantry part 301, during movement of imaging system 227, and during image capture, couch 214 is controlled by the control system to remain stationary. The patient, to the extent possible, is also controlled during this time to remain stationary on couch.

Following image capture, support structure 232 and thus imaging system 227 are controlled to move in the direction of arrow 248 to enable the gantry to be repositioned for treatment of an irradiation target at treatment position 210. Control over movement of support structure 232 and thus imaging system 227 in the direction of arrow 248 following image capture is as described with respect to FIGS. 24 and 25.

Also following image capture, gantry part 301 is controlled to move in the direction of arrow 305 toward treatment position 210 and also to reconnect to gantry part 306. This movement results in basically the same the gantry configuration shown in FIG. 24. To implement the movement, the control system may instruct motor 324 to move gantry part 301 toward treatment position 210 and the amount of that movement. In general, the amount of movement in the direction of arrow 305 should be equal and opposite to the amount of movement in the direction of arrow 304. To implement movement in the direction of arrow 305, motor 324 generates enough force to cause connectors 311a on gantry part 301 to reengage their counterpart connectors 311b on gantry part 306. In some implementations, the control system need not instruct the amount of movement back toward the accelerator, but rather may monitor the connectors and stop movement when the connectors have reengaged.

Referring to FIG. 26, in an example of sequential movement, gantry part 301 may move away from the treatment position 210 in the direction of arrow 304 to position 320. Then, support structure 232 may move the imaging system from its original position 250 (FIG. 24) into treatment position 210 in the direction of arrow 247. Then, after the imaging system performs image capture operations, support structure 232 may move the imaging system 227 away from treatment position 210 in the direction of arrow 248 back to its original position 250 shown in FIG. 24. Then, gantry part 301 may move in the direction of arrow 305 back so that it aligns to treatment position 210 and connects to gantry part 306, producing a configuration similar to that of FIG. 24.

In a concurrent movement scenario, gantry part 301 moves in the direction of arrow 304 to position 320 at the same time, or during at least part of the same time as, as support structure 232 moves imaging system 227 in the direction of arrow 247 to treatment position 210. Following image capture by the imaging system, gantry part 301 moves in the direction of arrow 305 to align with treatment position 210 at the same time as, or during at least part of the same time as, support structure 232 moves imaging system 227 in the direction of arrow 248 to its original position 250 (FIG. 24). Gantry part 301 and support structure 232 may move at the same speed or their speeds may be controlled to ensure that the gantry part and the support structure do not collide during movement.

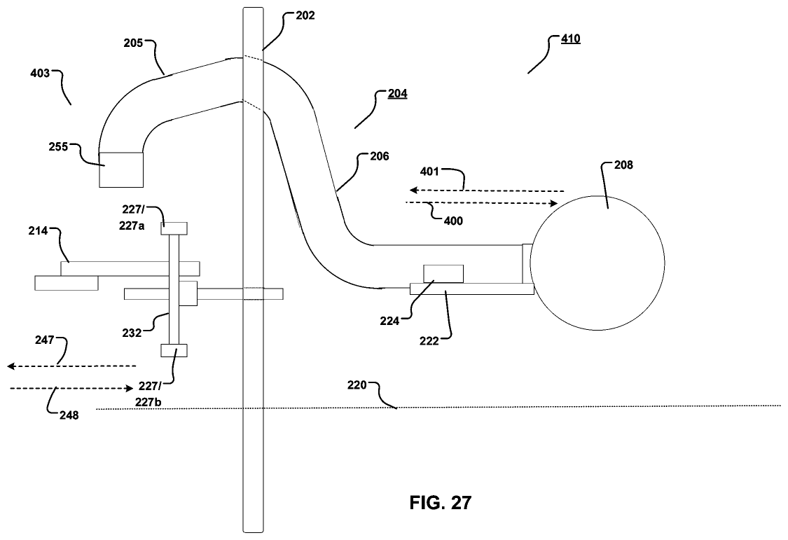

In the example particle therapy system 410 of FIG. 27, both beamline structure 204 and accelerator 208 are controlled to move in the directions of arrows 400 and 401. This configuration is advantageous in that it need not disconnect and reconnect the gantry from the accelerator. To implement movement the beamline structure 204 and accelerator 208 together—that is, in tandem—accelerator 208 may be connected physically to rail 222 or any other appropriate mechanism that enables translational movement of the accelerator. In some implementations, the connection between beamline structure 204 and accelerator 208 is strong enough that the translational movement of beamline structure 204 causes equal translational movement of accelerator 208. That is, the accelerator moves along with the gantry without detaching. In some implementations, an additional separate motor (not shown) may be connected to particle accelerator 208 to assist driving the particle accelerator 208 along rail 222.

To implement translational movement of beamline structure 204 and accelerator 208, the control system may instruct motor 224 to move beamline structure 204 and accelerator 208 in the direction of arrow 401 and the amount of that movement. In some implementations, the translational movement of beamline structure 204 and accelerator 208 in the direction of arrow 401, and later in the direction of arrow 400, is for at least 30 cm, for between 30 cm and 1 m, or for more than 1 m. In general, any appropriate amount of translational movement may be implemented.

Control over movement of support structure 232 and thus imaging system 227 in the direction of arrow 247 shown in FIG. 27 is as described with respect to FIGS. 24 and 25. During movement of beamline structure 204 and accelerator 208, during movement of imaging system 227, and during image capture, couch 214 is controlled by the control system to remain stationary. The patient, to the extent possible, is also controlled during this time to remain stationary on couch.

Following image capture, support structure 232 and thus imaging system 227 are controlled to move in the direction of arrow 248 to enable the gantry to be repositioned for treatment of an irradiation target at treatment position 210. Control over movement of support structure 232 and thus imaging system 227 in the direction of arrow 248 following image capture is as described with respect to FIGS. 24 and 25.

Also following image capture, beamline structure 204 and accelerator 208 are controlled to move in the direction of arrow 400. This movement results in basically the same the gantry and accelerator configuration shown in FIG. 24. To implement the movement, the control system may instruct motor 224 (and any other motor(s)) to move beamline structure 204 and accelerator 208 and the amount of that movement. In general, the amount of movement in the direction of arrow 400 should be equal and opposite to the amount of movement in the direction of arrow 401.

Referring to FIG. 27, in an example sequential movement, beamline structure 204 and accelerator 208 may move away from the treatment position 210 in the direction of arrow 401 to position 403. Then, support structure 232 may move the imaging system from its original position 250 (FIG. 24) to align with treatment position 210 in the direction of arrow 247. Then, after the imaging system performs image capture operations, support structure 232 may move the imaging system 227 away from treatment position 210 in the direction of arrow 248 back to its original position 250 shown in FIG. 24. Then, beamline structure 204 and accelerator 208 may move in the direction of arrow 400 so that nozzle 255 aligns to treatment position 210 as shown in FIG. 24.

In a concurrent movement scenario, beamline structure 204 and accelerator 208 move in the direction of arrow 401 to position 403 at the same time as, or during part of the same time as, support structure 232 moves imaging system 227 in the direction of arrow 247 to treatment position 210. Following image capture by the imaging system, beamline structure 204 and accelerator 208 move in the direction of arrow 400 so that nozzle 255 aligns to treatment position 210 at the same time as, or during part of the same time as, support structure 232 moves imaging system 227 in the direction of arrow 248 to its original position 250 shown in FIG. 24. Beamline structure 204/accelerator 208 and support structure 232 may move at the same speed or their speeds may be controlled to avoid collisions of system components.

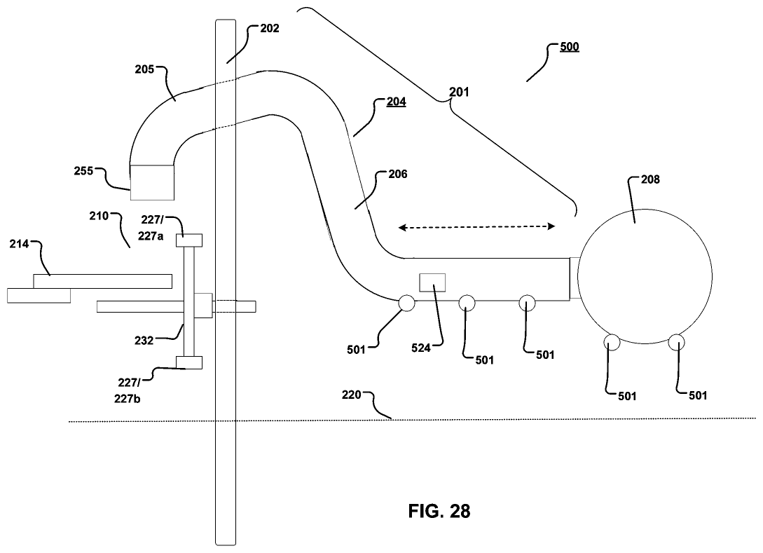

FIG. 28 shows a particle therapy system 500 of the type shown in FIG. 24. The mount in particle therapy system 500 includes collection(s) of wheels or rollers 501 instead of, or in addition to, the track or rail described with respe31ct to FIGS. 3124 to 27. The mount shown in FIG. 28, or components thereof (e.g., individual rollers) may be used in any of the configurations described herein to move the entirety of the beamline structure as shown in FIG. 24, part of the beamline structure as shown in FIG. 26, or both the accelerator and the beamline structure (or both the accelerator and part of the beamline structure) in tandem as shown in FIG. 26. Motor 524 may control operation of wheels or rollers 501 in accordance with commands from the control system.

The systems described herein are not limited to the mounts described herein; rather, any appropriate structure that moves, or enables movement of at least part of the gantry and/or the accelerator may be used.

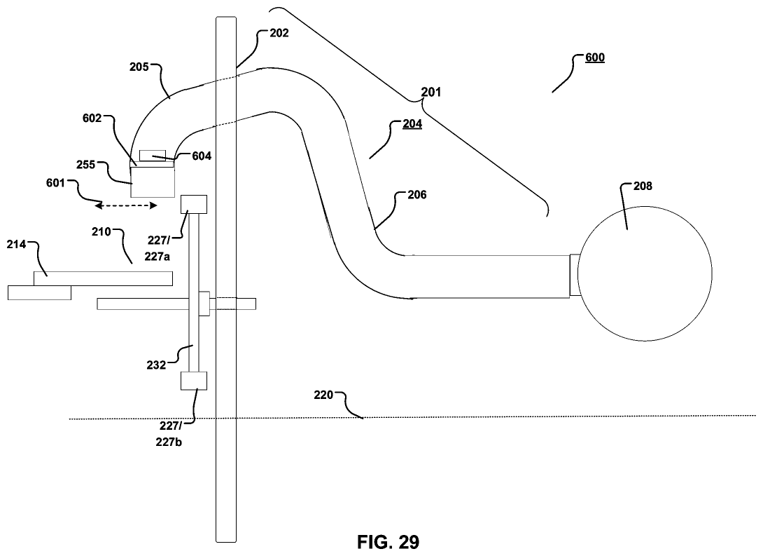

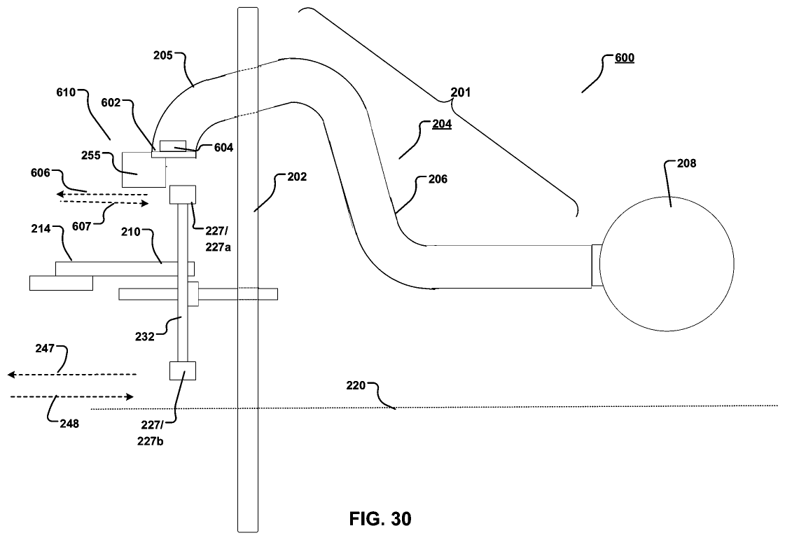

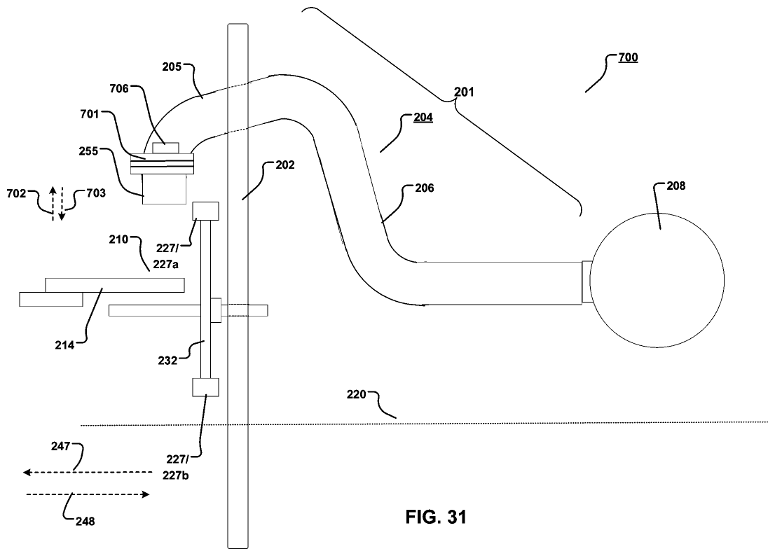

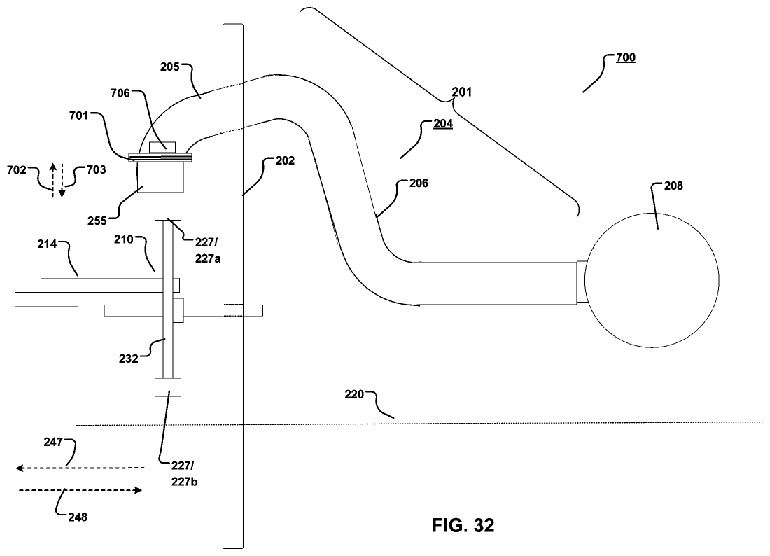

In the example of FIGS. 29 to 32, the part of the gantry/beamline structure that is configured to move is nozzle 255. For example, as shown in FIGS. 29 and 30, nozzle 255 may be configured to move along the longitudinal direction of the gantry—for example, parallel to the beamline or axis or rotation of the beamline—relative to the treatment position. For example, as shown in FIGS. 31 and 32, nozzle 255 may be configured to move perpendicular to the beamline or axis or rotation of the beamline relative to the treatment position. The movement shown in FIGS. 31 and 32 is another example type of translational movement than that shown in the other figures. The movements of the nozzle described with respect to FIGS. 29 to 32 may be combined with any of the movements of the rest of the beamline structure described with respect to FIGS. 24 to 28 in order to provide additional movement degrees of freedom.

Referring to FIGS. 29 and 30, in example particle therapy system 600, nozzle 255 may be part of a drawer that is configured to move translationally in the directions of arrows 601. For example, nozzle 255 may be mounted on a track 602 and controlled by a motor 603 to implement the translational movement. The motor may respond to instructions from the control system to control the movement of the nozzle. To implement translational movement of nozzle 255, the control system may instruct motor 604 to move nozzle 255 away from treatment position 210 in the direction of arrow 606 (FIG. 30) and the amount of that movement. In some implementations, the translational movement of nozzle 255 away from the treatment position 210 in the direction of arrow 606, and later back toward treatment position 210 in the direction of arrow 607, is on the order of tens of centimeters, e.g., 10 cm, 20 cm, 30 cm, 40 cm, and so forth. However, any appropriate movement may be implemented. As shown in FIG. 30, movement of nozzle 255 in the direction of arrow 606 moves the nozzle out of alignment with the treatment position, allowing the imaging system to move to, and into alignment with, the treatment position to capture images at the treatment position.

Control over movement of support structure 202 and thus imaging system 227 in the direction of arrow 247 shown in FIGS. 29 and 39 is as described with respect to FIGS. 24 and 25. As described above, during movement of nozzle 255, during movement of imaging system 227, and during image capture, couch 214 is controlled by the control system to remain stationary. The patient, to the extent possible, is also controlled during this time to remain stationary on couch.

Following image capture, support structure 232 and thus imaging system 227 are controlled to move in the direction of arrow 248 to enable nozzle 255 to be repositioned for treatment of an irradiation target at treatment position 210. Control over movement of support structure 232 and thus imaging system 227 in the direction of arrow 248 following image capture is as described with respect to FIGS. 24 and 25.

Also following image capture, the nozzle 255 is controlled to move in the direction of arrow 607 to reposition it over, and in alignment with, treatment position 210. This movement results in basically the same the gantry configuration shown in FIG. 24. To implement the movement, the control system may instruct motor 604 to move nozzle 255 toward treatment position 210 along rail 602 and the amount of that movement. In general, the amount of movement in the direction of arrow 607 should be equal and opposite to the amount of movement in the direction of arrow 606.

Referring to FIG. 30, in an example sequential movement, nozzle 255 may move away from the treatment position 210 in the direction of arrow 606 to position 610. Then, support structure 232 may move the imaging system from its original position 250 (FIG. 24) into alignment with treatment position 210 in the direction of arrow 247. Then, after the imaging system performs image capture operations, support structure 232 may move the imaging system 227 away from treatment position 210 in the direction of arrow 248 back to its original position 250 shown in FIG. 24. Then, nozzle 255 may move in the direction of arrow 607 so that nozzle 255 aligns to treatment position 210 as shown in FIG. 24 to enable treatment at the treatment position.

In a concurrent movement scenario, nozzle 255 moves in the direction of arrow 606 to position 610 at the same time as, or during part of the same time as, support structure 232 moves imaging system 227 in the direction of arrow 247 toward treatment position 210. Following image capture by the imaging system, nozzle 255 moves in the direction of arrow 607 so that nozzle 255 aligns to treatment position 210 at the same time as, or during part of the same time as, support structure 232 moves imaging system 227 in the direction of arrow 248 to its original position 250 shown in FIG. 24. Nozzle 255 may move at the same speed or support structure 232 or the speed of the two may be controlled by the control system to avoid collision.

Referring to FIGS. 31 and 32, in example particle therapy system 700, nozzle 255 may be connected to a telescopic mount 701 that is configured to move translationally in the directions of arrows 702, 703. For example, motor 706 may implement the translational movement. The motor may respond to instructions from the control system to control the movement of the nozzle toward or away from couch 214. FIG. 31 shows movement toward couch 214 and FIG. 32 shows movement away from couch 214.

To implement translational movement of nozzle 255, the control system may instruct motor 706 to move nozzle 255 relative to the couch and the amount of that movement. In some implementations, the translational movement of nozzle 255 away from the couch in the direction of arrow 702, and later back toward the couch in the direction of arrow 703, is on the order of tens of centimeters, e.g., 10 cm, 20 cm, 30 cm, 40 cm, and so forth. However, any appropriate movement may be implemented.

Control over movement of support structure 202 and thus imaging system 227 in the direction of arrow 247 shown in FIG. 31 is as described with respect to FIGS. 24 and 25. As described above, during movement of nozzle 255, during movement of imaging system 227, and during image capture, couch 214 is controlled by the control system to remain stationary. The patient, to the extent possible, is also controlled during this time to remain stationary on couch. The patient couch may also remain stationary.

Following image capture, support structure 232 and thus imaging system 227 are controlled to move in the direction of arrow 248 to enable nozzle 255 to be repositioned for treatment of an irradiation target at treatment position 210. Control over movement of support structure 232 and thus imaging system 227 in the direction of arrow 248 following image capture is as described with respect to FIGS. 24 and 25.

Also following image capture, nozzle 255 is controlled to move in the direction of arrow 703 toward patient couch 213 for treatment (FIG. 31). This movement results in basically the same the gantry configuration shown in FIG. 24. To implement the movement, the control system may instruct motor 706 to move nozzle 255 toward patient couch 214, and thus toward the treatment position, and the amount of that movement. In general, the amount of movement in the direction of arrow 703 should be equal and opposite to the amount of movement in the direction of arrow 702.

In an example sequential movement, nozzle 255 may move away from the treatment position 210 in the direction of arrow 702 to the configuration of FIG. 32. Then, support structure 232 may move the imaging system from its original position 250 (FIG. 24) into treatment position 210 in the direction of arrow 247. Then, after the imaging system performs image capture operations, support structure 232 may move the imaging system 227 away from treatment position 210 in the direction of arrow 248 back to its original position 250 shown in FIG. 24. Then, nozzle 255 may move in the direction of arrow 703 closer to treatment position 210 as shown in FIG. 31 In general, bringing the nozzle closer to the treatment position may reduce air-based beam spread and provide more accurate spot size and thus treatment.

In a concurrent movement scenario, nozzle 255 moves in the direction of arrow 702 to the position shown in FIG. 32 at the same time as, or during part of the same time as, support structure 232 moves imaging system 227 in the direction of arrow 247 to treatment position 210. Following image capture by the imaging system, nozzle 255 moves in the direction of arrow 702 toward the position shown in FIG. 31 at the same time as, or during part of the same time as, support structure 232 moves imaging system 227 in the direction of arrow 248 to its original position 250 shown in FIG. 24. Nozzle 255 may move at the same speed or support structure 232 or the speed of the two may be controlled by the control system in order to avoid collisions.

Another motivation for having a translating gantry is to extend the effective beam field size of the particle (e.g., proton) delivery. The beam field size, e.g., the largest region over which a proton system can deliver a single treatment field or beam without moving the patient for a single position of the gantry, may be limited by many design choices made during the design of the beamline such as the strength of the scanning magnet, the aperture of the beamline magnets, the size and range of travel of range shifter plates, and the range of travel of collimator elements in the collimator Adding one or more translating degree(s) of motion to the gantry may effectively extend this field size, allowing for protons to be delivered in one part of a field with the gantry at one position, then to a different part of a field with the gantry in a different position. In this way, many of the beamline elements (e.g., bending magnets, focusing magnets, scanning magnets, range shifter plates, automated collimator motion axes) may be designed for a smaller beam field size. In many cases this would reduce the cost, size, and complexity of these beamline elements.

Extending the effective field size may also improve the treatment functionality of the particle therapy system, as many treatments are better suited for larger field sizes. For example, protons are often used to deliver craniospinal irradiations in which a patient's entire brain and spinal column are all treated. Such a treatment is too long in one direction to fit inside a typical field and these are often treated by many fields stitched together, with patient motion on the couch between fields. A translating gantry would allow such a field to be delivered without patient motion.

Translational gantry movement to implement an effective increase in the size of the beam field is described with respect to the configuration of FIG. 24; however, translational gantry movement to implement an effective increase in the size of the beam field may be implemented using any appropriate configuration described herein that implements translational movement, or combinations thereof.

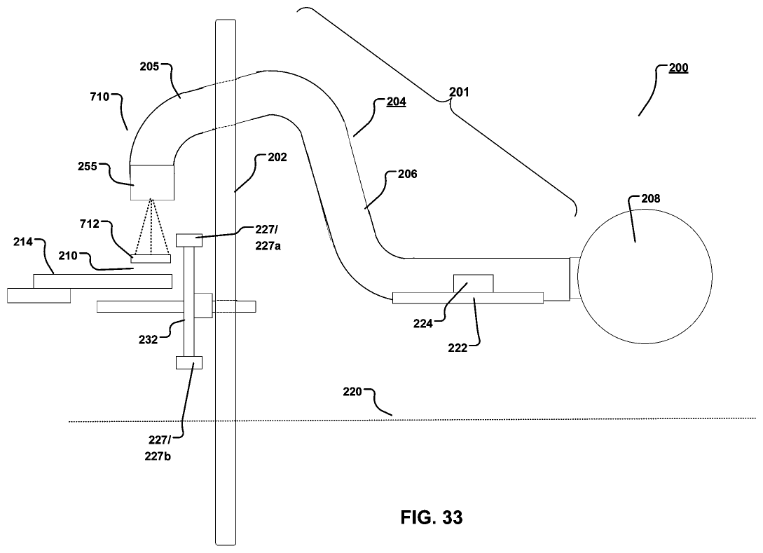

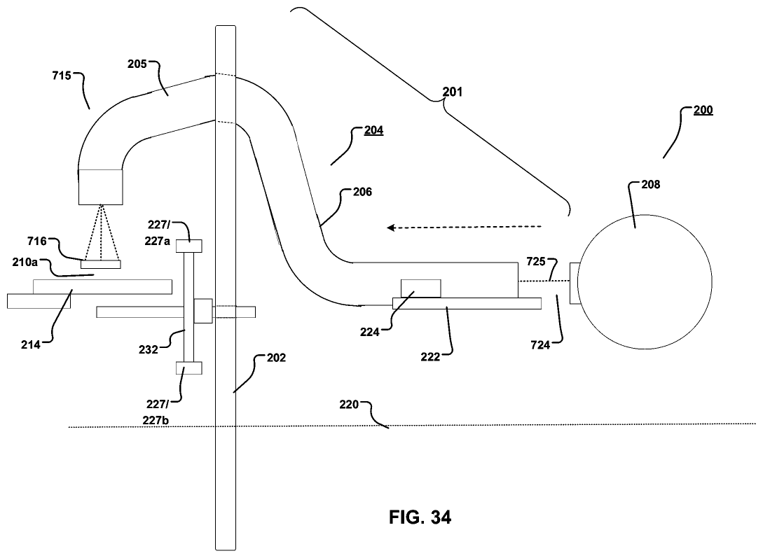

FIG. 33 shows beamline structure 204 in a first position 710, which produces a first beam field 712. FIG. 34 shows beamline structure 204 in a second position 715 following translational movement, which produces a second beam field 716. In this second position, gantry 201 may be disconnected from accelerator 208, producing an air gap 724 between the two that is traversed by the particle beam 725 during treatment. First beam field 712 and beam field 716 may overlap or not overlap. In any case, the translational movement of the gantry extends the range over which treatment can be provided without moving the couch or the patient on the couch.

FIGS. 33 and 34 show the gantry in only different two positions. However, at least part of the gantry may be translationally movable into three, four, five, or more positions, e.g., for a single rotational position, to provide further increases in the effective size of the beam field. In any case, the resulting effective size of the beam field may be greater than the predefined size of the beam field that can be produced without translational movement of the gantry. The effective size of the beam field may be 1.5 times greater than the predefined size, 2 times greater than the predefined size, 3 times greater than the predefined size, 4 times greater than the predefined size, 5 times greater than the predefined size, 6 times greater than the predefined size, 7 times greater than the predefined size, or more based on the amount that the gantry can be moved and the granularity of the movement.

Any of the features described with respect to FIGS. 24 to 34 may be combined. For example, to increase the effective beam field, only part of the gantry need be moved as shown in FIG. 26, or both the gantry and the accelerator may be moved as shown in FIG. 27. In another example, the nozzle movement of FIGS. 29 to 32 may be combined with any gantry movement of FIGS. 24 to 28, 33, and 34. In still another example, while the patient is being treated with the gantry using beam field 716 (which may be the first beam field used for treatment), the imaging system may be moved into position over treatment position 210 to perform imaging at the same time as treatment is performed.

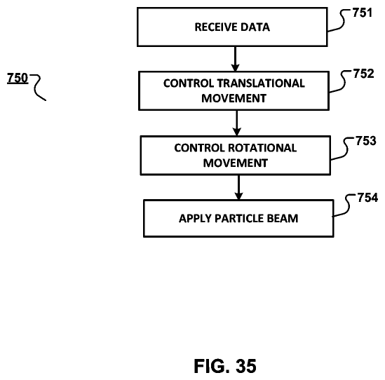

FIG. 35 shows an example operational sequence 750 for controlling beam field size. The control system receives (751) data representing a size of a target beam field. This data may be input by a user or part of a treatment plan supplied to the control system. The control system controls (752) the motors and/or other components described herein to implement two or more translational movements of at least part of a gantry (e.g., at least part of the beamline structure) relative to an irradiation target based on the data. At each of discrete translational position, if necessary, the control system controls (753) the motors and/or other components described herein to implement rotational movement of at least part of the gantry (e.g., the output channel) based on the data to position the nozzle to treat the irradiation target. The control system controls the particle accelerator to apply (754) particle beam to the irradiation target at each discrete position at the specified rotational positions based on the treatment plan. During translational movement, the rotational movement, and treatment, the couch and patient are controlled to remain stationary. The individual beam fields at each translational position of the beamline structure combine to produce the target beam field.

Clinical users may prefer high-quality volumetric imaging of the patient with the patient positioned at or near their treatment positions. This may reduce the amount of patient motion required between imaging and treating. In many proton therapy systems with treatment gantries, the gantry and nozzle that is mounted on it may get close to the patient, limiting the amount of space available for an imager. When the gantry can translate as described herein, it can be moved out of the way so that an imaging system can be deployed into the treatment space. In this way, the patient on the couch can be positioned near their first treatment position, the gantry can be translated out of the way, the imager deployed, and images acquired, the imaging system stowed, and the gantry returned to treatment position, image-based corrections applied, and treatment delivered. Of particular interest is for an imaging device, for example a diagnostic CT scanner, to have a very fast scan speed (image acquisition in less than 10 s for example) and axis of rotation coaxial with the gantry rotation axis.

In some implementations, imaging may be performed before and/or during treatment to identify a target location within the patient and/or to control operation of the gantry and scanning in order to direct the particle beam to the irradiation target in the patient. An example imaging system may include one or more of: a computerized tomography (CT) scanner, a two-dimensional (2D) X-ray device, a magnetic resonance imaging (MRI) device, a fan-beam CT scanner, a 2D camera, a three-dimensional (3D) camera, a surface imaging device, or a cone-beam CT scanner

In some implementations, two 2D imaging devices are mounted to support structure 232 in orthogonal planes to enable 2D image-guided radiation therapy (IGRT). IGRT includes the use of imaging during radiation treatment to improve the precision and accuracy of treatment delivery. IGRT may be used to treat tumors in areas of the body that move, such as the lungs. The 2D imaging devices can be rotated to enable cone-beam CT imaging, including simultaneously acquired dual energy imaging. The imaging devices may also, or alternatively, include an X-ray source and an image panel for cone-beam CT image acquisition or a fan-beam diagnostic quality CT imaging device. Alternatively, one plane may include a cone-beam CT imaging device and another plane may include a fan-beam diagnostic quality CT imaging device.

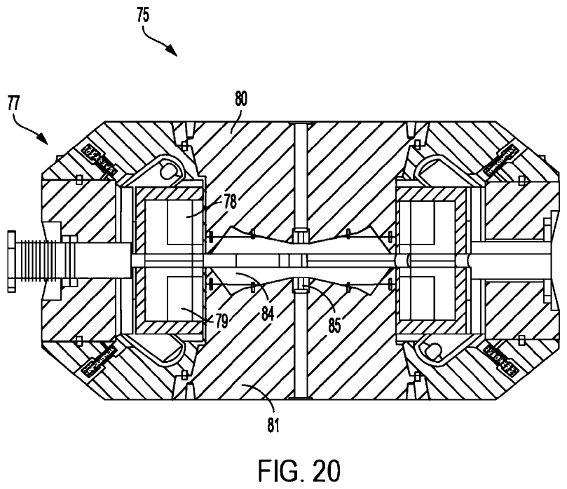

As described herein, an example proton therapy system scans a proton beam in three dimensions across an irradiation target in order to destroy malignant tissue. FIG. 20 shows a cross-section of components 75 of an example superconducting synchrocyclotron that may be used to provide a particle (e.g., a proton) beam in the proton therapy system. In this example, components 75 include a superconducting magnet 77. The superconducting magnet includes superconducting coils 78 and 79. The superconducting coils are formed of multiple integrated conductors, each of which includes superconducting strands—for example, four strands or six strands—wound around a center strand which may itself be superconducting or non-superconducting. Each of the superconducting coils 78, 79 is for conducting a current that generates a magnetic field (B). The magnetic yokes 80, 81 or smaller magnetic pole pieces shape that magnetic field in a cavity 84 in which particles are accelerated. In an example, a cryostat (not shown) uses liquid helium (He) to conductively cool each coil to low-temperature superconducting temperatures, e.g., around 4° Kelvin (K).

In some implementations, the particle accelerator includes a particle source 85, such as a Penning Ion Gauge (PIG) source, to provide an ionized plasma column to cavity 84. Hydrogen gas, or a combination of hydrogen gas and a noble gas, is ionized to produce the plasma column. A voltage source provides a varying radio frequency (RF) voltage to cavity 84 to accelerate particles from the plasma column within the cavity. As noted, in an example, the particle accelerator is a synchrocyclotron. Accordingly, the RF voltage is swept across a range of frequencies to account for relativistic effects on the particles, such as increasing particle mass, when accelerating particles within the acceleration cavity. The RF voltage drives a dee plate contained within the cavity and has a frequency that is swept downward during the accelerating cycle to account for the increasing relativistic mass of the protons and the decreasing magnetic field. A dummy dee plate acts as a ground reference for the dee plate. The magnetic field produced by running current through the superconducting coils, together with sweeping RF voltage, causes particles from the plasma column to accelerate orbitally within the cavity and to increase in energy as a number of turns increases. The particles in the outermost orbit are directed to an extraction channel (not shown) and are output from the synchrocyclotron as a particle beam. In a synchrocyclotron, the particle beam is pulsed such that bunches of particles are output periodically.

The magnetic field in the cavity is shaped to cause particles to move orbitally within the cavity as described above. The example synchrocyclotron employs a magnetic field that is uniform in rotation angle and falls off in strength with increasing radius. In some implementations, the maximum magnetic field produced by the superconducting (main) coils may be within the range of 2.5 T to 20 T at a center of the cavity, which falls off with increasing radius. For example, the superconducting coils may be used in generating magnetic fields at, or that exceed, one or more of the following magnitudes: 2.5 T, 3.0 T, 3.1 T, 3.2 T, 3.3 T, 3.4 T, 3.5 T, 3.6 T, 3.7 T, 3.8 T, 3.9 T, 4.0 T, 4.1 T, 4.2 T, 4.3 T, 4.4 T, 4.5 T, 4.6 T, 4.7 T, 4.8 T, 4.9 T, 5.0 T, 5.1 T, 5.2 T, 5.3 T, 5.4 T, 5.5 T, 5.6 T, 5.7 T, 5.8 T, 5.9 T, 6.0 T, 6.1 T, 6.2 T, 6.3 T, 6.4 T, 6.5 T, 6.6 T, 6.7 T, 6.8 T, 6.9 T, 7.0 T, 7.1 T, 7.2 T, 7.3 T, 7.4 T, 7.5 T, 7.6 T, 7.7 T, 7.8 T, 7.9 T, 8.0 T, 8.1 T, 8.2 T, 8.3 T, 8.4 T, 8.5 T, 8.6 T, 8.7 T, 8.8 T, 8.9 T, 9.0 T, 9.1 T, 9.2 T, 9.3 T, 9.4 T, 9.5 T, 9.6 T, 9.7 T, 9.8 T, 9.9 T, 10.0 T, 10.1 T, 10.2 T, 10.3 T, 10.4 T, 10.5 T, 10.6 T, 10.7 T, 10.8 T, 10.9 T, 11.0 T, 11.1 T, 11.2 T, 11.3 T, 11.4 T, 11.5 T, 11.6 T, 11.7 T, 11.8 T, 11.9 T, 12.0 T, 12.1 T, 12.2 T, 12.3 T, 12.4 T, 12.5 T, 12.6 T, 12.7 T, 12.8 T, 12.9 T, 13.0 T, 13.1 T, 13.2 T, 13.3 T, 13.4 T, 13.5 T, 13.6 T, 13.7 T, 13.8 T, 13.9 T, 14.0 T, 14.1 T, 14.2 T, 14.3 T, 14.4 T, 14.5 T, 14.6 T, 14.7 T, 14.8 T, 14.9 T, 15.0 T, 15.1 T, 15.2 T, 15.3 T, 15.4 T, 15.5 T, 15.6 T, 15.7 T, 15.8 T, 15.9 T, 16.0 T, 16.1 T, 162 T, 16.3 T, 16.4 T, 16.5 T, 16.6 T, 16.7 T, 16.8 T, 16.9 T, 17.0 T, 17.1 T, 17.2 T, 17.3 T, 17.4 T, 17.5 T, 17.6 T, 17.7 T, 17.8 T, 17.9 T, 18.0 T, 18.1 T, 18.2 T, 18.3 T, 18.4 T, 185 T, 18.6 T, 18.7 T, 18.8 T, 18.9 T, 19.0 T, 19.1 T, 19.2 T, 19.3 T, 19.4 T, 19.5 T, 19.6 T, 19.7 T, 19.8 T, 19.9 T, 20.0 T, 20.1 T, 20.2 T, 20.3 T, 20.4 T, 20.5 T, 20.6 T, 20.7 T, 20.8 T, 20.9 T, or more. Furthermore, the superconducting coils may be used in generating magnetic fields that are outside the range of 2.5 T to 20 T or that are within the range of 3 T to 20 T but that are not specifically listed herein.

By generating a high magnetic field having a magnitude such as those described above, the bend radius of particles orbiting within cavity 84 can be reduced. As a result of the reduction in the bend radius, a greater number of particle orbits can be made within a given-sized cavity. So, the same number of orbits can be fit within a smaller cavity. Reducing the size of the cavity reduces the size of the particle accelerator in general, since a smaller cavity requires smaller magnetic yokes or pole pieces, among other components. In some implementations, the size or volume of the particle accelerator may be 4 m³ or less, 3 m³ or less, or 2 m³ or less.

In some implementations, such as the implementations shown in FIG. 20, the relatively large ferromagnetic magnetic yokes 80, 81 act as magnetic returns for stray magnetic fields produced by the superconducting coils. In some systems, a magnetic shield (not shown) surrounds the yokes. The return yokes and the shield together act to reduce stray magnetic fields, thereby reducing the possibility that stray magnetic fields will adversely affect the operation of the particle accelerator.

In some implementations, the return yokes and/or shield may be replaced by, or augmented by, an active return system. An example active return system includes one or more active return coils that conduct current in a direction opposite to current through the main superconducting coils. In some implementations, there is an active return coil for each superconducting main coil, e.g., two active return coils—one for each main superconducting coil. Each active return coil may also be a superconducting coil that surrounds the outside of a corresponding main superconducting coil concentrically. In some implementations, the active return coils may be or include non-superconducting coils. By using an active return system, the relatively large ferromagnetic magnetic yokes 80, 81 can be replaced with magnetic pole pieces that are smaller and lighter. Accordingly, the size and weight of the synchrocyclotron can be reduced further without sacrificing performance. An example of an active return system that may be used is described in U.S. Pat. No. 8,791,656 (Zwart) entitled “Active Return System”. The content of U.S. Pat. No. 8,791,656, particularly the content related to the return coil configuration (e.g., FIGS. 2, 4, and 5 of U.S. Pat. No. 8,791,656 and the accompanying description), is incorporated herein by reference.

Another example of a particle accelerator that may be used in the particle therapy system herein is described in U.S. Pat. No. 8,975,836 (Bromberg) entitled “Ultra-Light Magnetically Shielded High-Current, Compact Cyclotron”. The content of U.S. Pat. No. 8,975,836, particularly the content related to “cyclotron 11” or “iron-free cyclotron 11” of FIGS. 4, 17 and 18 of U.S. Pat. No. 8,975,836 and the accompanying description, is incorporated herein by reference.

In some implementations, a synchrocyclotron used in the proton therapy system described herein may be a variable-energy synchrocyclotron. In some implementations, a variable-energy synchrocyclotron is configured to vary the energy of the output particle beam by varying the magnetic field in which the particle beam is accelerated. For example, the current may be set to any one of multiple values to produce a corresponding magnetic field. For example, the current may be set to one of two values to produce the dual-energy particle accelerator described previously. In an example implementation, one or more sets of superconducting coils receives variable electrical current to produce a variable magnetic field in the cavity. In some examples, one set of coils receives a fixed electrical current, while one or more other sets of coils receives a variable current so that the total current received by the coil sets varies. In some implementations, all sets of coils are superconducting. In some implementations, some sets of coils, such as the set for the fixed electrical current, are superconducting, while other sets of coils, such as the one or more sets for the variable current, are non-superconducting (e.g., copper) coils.

Generally, in a variable-energy synchrocyclotron, the magnitude of the magnetic field is scalable with the magnitude of the electrical current. Adjusting the total electric current of the coils in a predetermined range can generate a magnetic field that varies in a corresponding, predetermined range. In some examples, a continuous adjustment of the electrical current can lead to a continuous variation of the magnetic field and a continuous variation of the output beam energy. Alternatively, when the electrical current applied to the coils is adjusted in a non-continuous, step-wise manner, the magnetic field and the output beam energy also varies accordingly in a non-continuous (step-wise) manner. The step-wise adjustment can produce the dual energies described previously. In some implementations, each step is between 10 MeV and 80 MeV in size. The scaling of the magnetic field to the current can allow the variation of the beam energy to be carried out relatively precisely, thus reducing the need for an energy degrader. An example of a variable-energy synchrocyclotron that may be used in the particle therapy systems described herein is described in U.S. Pat. No. 9,730,308 entitled “Particle Accelerator That Produces Charged Particles Having Variable Energies”. The content U.S. Pat. No. 9,730,308 is incorporated herein by reference, particularly the content that enables operation of a synchrocyclotron at variable energies, including the content described in columns 5 through 7 of U.S. Pat. No. 9,730,308 and FIG. 13 and its accompanying description.

In implementations of the particle therapy system that use a variable-energy synchrocyclotron, controlling the energy of the particle beam to treat a portion of the irradiation target may be performed in accordance with the treatment plan by changing the energy of the particle beam output by the synchrocyclotron. In such implementations, an energy degrader may or may not be used. For example, controlling the energy of the particle beam may include setting the current in the synchrocyclotron main coils to one of multiple values, each which corresponds to a different energy at which the particle beam is output from the synchrocyclotron. An energy degrader may be used along with a variable-energy synchrocyclotron to provide additional changes in energy, for, example, between discrete energy levels provided by the synchrocyclotron.

The particle therapy system and its variations described herein may be used to apply ultra-high dose rates of radiation—so called, “FLASH” dose rates of radiation—to an irradiation target in a patient. In this regard, experimental results in radiation therapy have shown an improvement in the condition of healthy tissue subjected to radiation when the treatment dose is delivered at ultra-high (FLASH) dose rates. In an example, when delivering doses of radiation at 10 to 20 Gray (Gy) in pulses of less than 500 milliseconds (me) reaching effective dose rates of 20 to 100 Gray-per-second (Gy/S), healthy tissue experiences less damage than when irradiated with the same dose over a longer time scale, while tumors are treated with similar effectiveness. A theory that may explain this “FLASH effect” is based on the fact that radiation damage to tissue is proportionate to oxygen supply in the tissue. In healthy tissue, the ultra-high dose rate radicalizes the oxygen only once, as opposed to dose applications that radicalize the oxygen multiple times over a longer timescale. This may lead to less damage in the healthy tissue using the ultra-high dose rate.

In some examples, as noted above, ultra-high dose rates of radiation may include doses of radiation that exceed 1 Gray-per-second for a duration of less than 500 ms. In some examples, ultra-high dose rates of radiation may include doses of radiation that exceed 1 Gray-per-second for a duration that is between 10 ms and 5 s. In some examples, ultra-high dose rates of radiation may include doses of radiation that exceed 1 Gray-per-second for a duration that is less than 5 s.

In some examples, ultra-high dose rates of radiation include doses of radiation that exceed one of the following doses for a duration of less than 500 ms: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second. In some examples, ultra-high dose rates of radiation include doses of radiation that exceed one of the following doses for a duration that is between 10 ms and 5 s: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second. In some examples, ultra-high dose rates of radiation include doses of radiation that exceed one of the following doses for a duration that is less than 5 s: 2 Gray-per-second, 3 Gray-per-second, 4 Gray-per-second, 5 Gray-per-second, 6 Gray-per-second, 7 Gray-per-second, 8 Gray-per-second, 9 Gray-per-second, 10 Gray-per-second, 11 Gray-per-second, 12 Gray-per-second, 13 Gray-per-second, 14 Gray-per-second, 15 Gray-per-second, 16 Gray-per-second, 17 Gray-per-second, 18 Gray-per-second, 19 Gray-per-second, 20 Gray-per-second, 30 Gray-per-second, 40 Gray-per-second, 50 Gray-per-second, 60 Gray-per-second, 70 Gray-per-second, 80 Gray-per-second, 90 Gray-per-second, or 100 Gray-per-second.

In some examples, ultra-high dose rates of radiation include doses of radiation that exceed one or more of the following doses for a duration of less than 500 ms, for a duration that is between 10 ms and 5 s, or for a duration that is less than 5 s: 100 Gray-per-second, 200 Gray-per-second, 300 Gray-per-second, 400 Gray-per-second, or 500 Gray-per-second.

In some examples, ultra-high dose rates of radiation include doses of radiation that are between 20 Gray-per-second and 100 Gray-per-second for a duration of less than 500 ms. In some examples, ultra-high dose rates of radiation include doses of radiation that are between 20 Gray-per-second and 100 Gray-per-second for a duration that is between 10 ms and 5 s. In some examples, ultra-high dose rates of radiation include doses of radiation that are between 20 Gray-per-second and 100 Gray-per-second for a duration that is less than 5 s. In some examples, ultra-high dose rate rates of radiation include doses of radiation that are between 40 Gray-per-second and 120 Gray-per-second for a time period such as less than 5 s. Other examples of the time period are those provided above.

In some implementations, the particle therapy systems may treat three-dimensional columns of the target using ultra-high dose rate radiation—the FLASH doses of radiation. These systems scale the ultra-high dose rate deliveries to targets using pencil beam scanning. In some examples, pencil beam scanning includes delivering a series of small beams of particle radiation that can each have a unique direction, energy, and charge. By combining doses from these individual beams, a three-dimensional target treatment volume may be treated with radiation. Furthermore, instead of organizing the treatment into layers at constant energies, the systems organize the treatment into columns defined by the direction of a stationary beam. The direction of the beam may be toward the surface of the target.

In some implementations, all or part of a column is treated before the particle beam is directed along another path through the irradiation target. In some implementations, a path through the target is all or part-way through the target. In an example, the particle beam may be directed along a path through a target and not deviate from that path. While directed along that path, the energy of the particle beam is changed. The particle beam does not move as its energy changes and, as a result, the particle beam treats all or a part of an interior portion of the target that extends along a length of the particle beam and along a width of the beam spot. The treatment is thus depth-wise along a longitudinal direction of the beam. For example, a portion of the target treated may extend from a spot of the beam at the surface of the target down through all or part of an interior of the target. The result is that the particle beam treats a three-dimensional columnar portion of the target using an ultra-high dose rate of radiation. In some examples, the particle beam may never again be directed along the same three-dimensional columnar portion more than once.

In some implementations, an irradiation target may be broken into micro-volumes. Although cubical micro-volumes may be used, the micro-volumes may have any appropriate shape, such as three-dimensional orthotopes, regular curved shapes, or irregular or amorphous shapes. In this example, each micro-volume is treated through delivery of FLASH radiation by column in the manner described herein. For example, column depths of a micro-volume may be treated with radiation by using energy degrader plates to change the beam energy or by controlling a variable-energy synchrocyclotron to change the beam energy. After an individual micro-volume has been treated, the next micro-volume is treated, and so forth until the entire irradiation target has been treated. Treatment of the micro-volumes may be in any appropriate order or sequence.

Examples of techniques for delivering FLASH doses that may be used in the particle therapy systems described herein are described in U.S. Patent Publication No. 2020/0298025 entitled “Delivery Of Radiation By Column And Generating A Treatment Plan Therefor”. The content of U.S. Patent Publication No. 2020/0298025 is incorporated herein by reference, particularly the content that describes delivering FLASH doses, including the content described with respect to FIGS. 11 to 19 and 30 to 43B and their accompanying description.

In some implementations, a particle accelerator other than a synchrocyclotron may be used in the particle therapy system described herein. For example, a cyclotron, a synchrotron, a linear accelerator, or the like may be substituted for the synchrocyclotron in the particle therapy systems described herein.

In some implementations, the scanning magnet(s) may be replaced with a scattering foil and the energy degrader may be a range modulator. In implementations such as this, the scattering foil scatters the particle beam across a treatment area and the depth to which the scattered beam is applied is controlled by the range modulator. The configurable collimator may remain in place to trim edges of the scattered beam.

Operation of the example particle therapy systems described herein, and operation of all or some component thereof, can be controlled, at least in part, using a control system 192 (FIG. 1) configured to execute one or more computer program products, e.g., one or more computer programs tangibly embodied in one or more non-transitory machine-readable media, for execution by, or to control the operation of, one or more data processing apparatus, e.g., a programmable processor, a computer, multiple computers, and/or programmable logic components. The control system may be segregated from the rest of the particle therapy system and/or it may be distributed at various locations, including on the particle therapy system.

All or part of the systems described in this specification and their various modifications may be configured or controlled at least in part by one or more computers such as the control system 192 using one or more computer programs tangibly embodied in one or more information carriers, such as in one or more non-transitory machine-readable storage media. A computer program can be written in any form of programming language, including compiled or interpreted languages, and it can be deployed in any form, including as a stand-alone program or as a module, part, subroutine, or other unit suitable for use in a computing environment. A computer program can be deployed to be executed on one computer or on multiple computers at one site or distributed across multiple sites and interconnected by a network.

Actions associated with configuring or controlling the systems described herein can be performed by one or more programmable processors executing one or more computer programs to control or to perform all or some of the operations described herein. All or part of the systems and processes can be configured or controlled by special purpose logic circuitry, such as, an FPGA (field programmable gate array) and/or an ASIC (application-specified integrated circuit) or embedded microprocessor(s) localized to the instrument hardware.

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, such as magnetic, magneto-optical disks, or optical disks. Non-transitory machine-readable storage media suitable for embodying computer program instructions and data include all forms of non-volatile storage area, including by way of example, semiconductor storage area devices, such as EPROM (erasable programmable read-only memory), EEPROM (electrically erasable programmable read-only memory), and flash storage area devices; magnetic disks, such as internal hard disks or removable disks; magneto-optical disks; and CD-ROM (compact disc read-only memory) and DVD-ROM (digital versatile disc read-only memory).

Elements of different implementations described may be combined to form other implementations not specifically set forth previously. Elements may be left out of the systems described previously without adversely affecting their operation or the operation of the system in general. Furthermore, various separate elements may be combined into one or more individual elements to perform the functions described in this specification.

Other implementations not specifically described in this specification are also within the scope of the following claims.

What is claimed is:

1. A system comprising:
a gantry comprising a beamline structure configured to direct a particle beam from an output of a particle accelerator toward an irradiation target at a treatment position, the beamline structure comprising magnetic bending elements to bend the particle beam along at least part of a length of the beamline structure; and
a mount on which at least part of the beamline structure is held, the mount being configured to enable translational movement of the at least part of the beamline structure relative to the irradiation target.

2. The system of claim 1, wherein the translational movement comprises movement along a longitudinal dimension of the gantry.

3. The system of claim 1, wherein the translational movement comprises movement toward or away from the particle accelerator.

4. The system of claim 1, further comprising:
the particle accelerator;
wherein the mount is configured to enable movement of the particle accelerator along with the at least part of the beamline structure.

5. The system of claim 1, wherein the mount is configured to enable movement of an entirety of the beamline structure relative to the irradiation target.

6. The system of claim 5, wherein the mount is configured to enable movement of the entirety of the beamline structure along a longitudinal dimension of the gantry.

7. The system of claim 5, wherein the mount is configured to enable movement of the entirety the beamline structure toward or away from the particle accelerator along at least part of a beamline of the particle beam.

8. The system of claim 1, wherein the translational movement causes the at least part of the beamline structure to move away from the particle accelerator and to produce an air gap between the at least part of the beamline structure and the particle accelerator, the particle beam to traverse the air gap from the particle accelerator to the at least part of the beamline structure.

9. The system of claim 1, wherein the at least part of the beamline structure is a first part of the beamline structure, the beamline structure comprising the first part and a second part of the beamline structure; and
wherein the translational movement causes the first part to move away from the second part and to produce an air gap between the first part and the second part, the particle beam to traverse the air gap from the second part to the first part.

10. The system of claim 9, wherein the second part is attached to the particle accelerator and is not movable relative to the particle accelerator.

11. The system of claim 1, wherein the at least part of the beamline structure comprises an output channel, the output channel comprising magnetic dipoles arranged in series to bend the particle beam by at least 90°;
wherein the gantry comprises a ring structure on which the output channel is mounted for rotation around the irradiation target; and
wherein the translational movement is parallel to an axis of rotation about which the output channel rotates on the ring structure.

12. The system of claim 1, wherein the translational movement is for at least 30 centimeters.

13. The system of claim 1, wherein the translational movement is between 30 centimeters and 1 meter.

14. The system of claim 1, further comprising:
an imaging system that is movable relative to the irradiation target; and
a control system to control the mount or the at least part of the gantry to move the at least part of the beamline structure away from a location proximate to the irradiation target, and to control movement of the imaging system toward the location;
wherein a couch holding the irradiation target is configured to remain stationary during movement of the imaging system and during movement of the mount or the at least part of the beamline structure.

15. The system of claim 14, wherein the mount is a first mount and the system comprises a second mount configured to enable rotational movement of the imaging system relative to the irradiation target; and
wherein the control system is configured to control movement of the imaging system by controlling translational movement of the second mount.

16. The system of claim 15, wherein the imaging system is rotatable around an axis of rotation defined by the second mount; and
wherein the translational movement of the second mount is parallel to the axis of rotation.

17. The system of claim 14, wherein the control system is configured to control movement of the imaging system away from the location and to control the mount or the at least part of the beamline structure to move the at least part of the beamline structure toward the location; and
wherein the couch holding the irradiation target is configured to remain stationary during movement of the imaging system and during movement of the mount or the at least part of the beamline structure.

18. The system of claim 17, wherein the mount is a first mount and the system comprises a second mount configured to enable rotational movement of the imaging system relative to the irradiation target; and
wherein the control system is configured to control movement of the imaging system by controlling translational movement of the second mount.

19. The system of claim 18, wherein the imaging system is rotatable around an axis of rotation defined by the second mount; and
wherein the translational movement of the second mount is parallel to the axis of rotation.

20. The system of claim 1, wherein the mount comprises one or more rails, the one or more rails being moveable or the at least part of the beamline structure being movable along the one or more rails.

21. The system of claim 1, wherein the mount comprises one or more rollers or wheels connected to the at least part of the beamline structure.

22. The system of claim 1, wherein the at least part of the beamline structure comprises a nozzle, the nozzle holding at least one of an energy degrader or a collimator.

23. The system of claim 22, further comprising:
an imaging system that is movable relative to the irradiation target; and
a control system to control the mount or the nozzle to move the nozzle away from a location proximate to the irradiation target, and to control movement of the imaging system toward the location;
wherein a couch holding the irradiation target is configured to remain stationary during movement of the imaging system and during movement of the mount or the nozzle.

24. The system of claim 22, wherein the mount comprises a rail-mounted drawer.

25. The system of claim 22, wherein the mount is configured to move the nozzle telescopically.

26. A method implemented on a particle therapy system, the method comprising:
receiving data representing a size of a target beam field;
controlling translational movement of at least part of a beamline structure of a gantry in the particle therapy system relative to an irradiation target based on the data, the beamline structure being configured to direct a particle beam from an output of a particle accelerator toward the irradiation target, the beamline structure comprising magnetic bending elements to bend the particle beam along at least part of a length of the beamline structure; and
controlling the particle accelerator to apply particle beam to the irradiation target at different translational positions of the at least part of the beamline structure based on the data, where a couch holding the irradiation target is to remain stationary during the translational movement of the at least part of the beamline structure and application of the particle beam.

27. The method of claim 26, further comprising:
controlling rotational movement of at least part of the beamline structure relative to the irradiation target, where the couch is controlled to remain stationary during the rotational movement of the at least part of the beamline structure.

28. The method of claim 26, wherein the translational movement comprises movement along a longitudinal dimension of the gantry to discrete positions along the irradiation target.

29. The method of claim 26, wherein the translational movement comprises movement toward or away from the particle accelerator along at least part of a beamline of the particle beam.

30. The method of claim 26, wherein the beamline structure comprises an output channel, the output channel comprising magnetic dipoles arranged in series to bend the particle beam by at least 90°;
wherein the gantry comprises a ring structure on which the output channel is mounted for rotation around the irradiation target; and
wherein the translational movement is parallel to an axis of rotation about which the output channel rotates on the ring structure.

31. The method of claim 26, further comprising:
controlling movement of an imaging system based on the translational movement of the at least part of the beamline structure while the irradiation target is controlled to remain stationary.

32. The method of claim 31, wherein the at least part of the beamline structure is controlled to move out of a predefined position and the imaging system is controlled to move to the predefined position following movement of the at least part of gantry.

33. The method of claim 32, wherein the imaging system is controlled to move out of the predefined position and the beamline structure is controlled to move to the predefined position following movement of the imaging system out of the predefined position.

34. The method of claim 26, wherein the size of a target beam field is greater than a size of a predefined beam field defined, at least in part, by the gantry absent the translational movement of the at least part of gantry.

35. The method of claim 34, wherein the size of a target beam field is at least 1.5 times the size of the predefined beam field.

36. The method of claim 34, wherein the size of a target beam field is at least twice the size of the predefined beam field.

37. The method of claim 34, wherein the size of a target beam field is at least five times the size of the predefined beam field.
